(19) United States
(12) Patent Application Publication
(10) Pub. No.: US 2026/0115200 A1
(43) Pub. Date: Apr. 30, 2026

(54) Treatments Using Methylthioninium Salt, Liquid Mineral Blends, and/or Secondary Physiologically Active Compounds With Physiological Therapies

(71) Applicant: Best 365 Labs Inc., Salt Lake City, UT (US)

(72) Inventors: Darren Lopez, West Valley City, UT (US); Dan Schmidt, Salt Lake City, UT (US)

(21) Appl. No.: 19/371,824

(22) Filed: Oct. 28, 2025

Related U.S. Application Data
(60) Provisional application No. 63/712,895, filed on Oct. 28, 2024.

(51) Int. Cl. A61K 31/5415; A61G 10/02; A61K 31/00; A61K 31/198; A61K 31/355; A61K 31/37; A61K 31/375; A61K 31/4415; A61K 31/455; A61K 31/46; A61K 31/465; A61K 31/4745; A61K 31/522; A61K 31/7084; A61K 33/08; A61K 33/14; A61K 33/32; A61K 33/38; A61K 36/67; A61K 36/77; A61K 36/82; A61N 2/00; A61N 5/06

(52) U.S. Cl. CPC A61K 31/5415; A61G 10/026; A61K 31/198; A61K 31/355; A61K 31/37; A61K 31/375; A61K 31/4415; A61K 31/455; A61K 31/46; A61K 31/465; A61K 31/4745; A61K 31/522; A61K 31/658; A61K 31/7084; A61K 33/08; A61K 33/14; A61K 33/32; A61K 33/38; A61K 36/67; A61K 36/77; A61K 36/82; A61N 2/00; A61N 5/06; A61N 2005/0659; A61N 2005/0662

(57) ABSTRACT

The present disclosure relates to systems and methods for protecting or enhancing a physiological function. The method includes orally co-administering to a methylthioninium salt, a liquid mineral blend of mineral salts/oxides/chlorides, and/or a secondary physiologically active compound to a subject, and subjecting the subject to a physiological therapy, such as thermal therapy, electromagnetic therapy, oxygenation therapy, or a combination thereof.

The present application claims the benefit of and priority to U.S. Provisional Patent Application No. 63/712,895, filed on Oct. 28, 2024, the entirety of which is incorporated herein by reference.

### BACKGROUND

Methylthioninium chloride, referred to conventionally as Methylene Blue, is a synthetic compound often used for treating and/or diagnosing certain medical conditions. It functions similarly to hemoglobin, which is a transporter of oxygen. Methylene blue is also a powerful redox agent, which is blue when oxidized and colorless when deoxygenated. In the therapeutic space, methylene blue has been used in the treatment of various types of conditions but may not be as effective as it could be, and thus, could benefit from co-formulation or coadministration of other cooperative or synergistic compounds and coordinated treatment protocols.

### DETAILED DESCRIPTION

In accordance with examples of the present disclosure, methylthioninium salt-containing compositions, methods of manufacture, and methods of treatment are provided herein that include co-administering methylthioninium salt with a second compound in combination with a physiological therapy that enhances the benefit of the methylthioninium salt, the second compound, or both. It may be said that the methylthioninium salt and/or the second compound can enhance the benefit of the physiological therapy, but either way, it is understood that there is an enhanced benefit that can be imparted on the methylthioninium salt, the second compound, and/or the physiological therapy due to the combination of treatments described herein. Example “second compound(s)” that can be co-administered or co-formulated with the methylthioninium salt, e.g., methylene blue, include any of a number of compounds that can enhance the benefit derived by a subject from consuming the methylthioninium salt, as described in greater detail hereinafter. Furthermore, examples of physiological therapies that can be used, include various thermal therapies, electromagnetic therapies, oxygenation therapies, etc., as described in greater detail hereinafter.

With respect to the coadministration of the methylthioninium salt and the second compound, in some examples, the methylthioninium salt-containing composition can be co-formulated with methylthioninium salt and a second compound. In other examples, the methylthioninium salt can be co-administered with the second compound, with each present within its own composition. Thus, coadministration of two or more compounds as described herein may be conducted by administering two or more active compounds as part of a single composition or formulation, or administering two or more active compounds at about the same time, e.g., within about 30 minutes of one another, from multiple compositions or formulations. Regardless, coadministration of the methylthioninium salt with the second compound can provide an additive or even synergistic benefit to the subject when administered properly within acceptable and safe dosage ranges. In further detail, by adding one or more of the physiological therapies described herein, additional benefits can be achieved in many instances. In examples herein, the methylthioninium salt-containing compositions and/or multiple compositions for delivering the methylthioninium salt and the second compound can be suitable for oral administration via oral ingestion (swallowing), e.g., capsule, tablet, soft-gel, powder, or liquid, e.g., solution or suspension, and/or via transmucosal oral administration by transmucosal delivery, e.g., buccal, sublabial, sublingual, etc. In some instances, oral delivery may include delivery occurring by oral ingestion with a portion being delivered by transmucosal oral delivery prior to or after swallowing the bulk of the composition, e.g., liquid suspensions may provide some transmucosal delivery due to contact with mucosal tissues within the mouth.

As used herein, the term “oral dosage form” refers to any of a number of oral delivery compositions or formulations, such as a tablet, capsule, soft-gel, powder, liquid suspension or solution, or transmucosal oral formulation. For example, a single tablet, a single liquid suspension shot, e.g., from 1-4 oz, or the like, refers to the oral dosage form. Furthermore, the term “daily dose” refers to the dosage given to a subject in a given day, either at a single point in time or divided over multiple doses during the day, and thus, can include the use of one or multiple oral dosage forms, e.g., 2 tablets, 3 capsules, 1 soft-gel capsule, etc. The term “daily” does not infer that a subject must take the dosage every single day, but rather refers to how much should be given as a daily dose for any single day. Thus, the methylthioninium salt-containing compositions can be prepared in oral dosage form, e.g., a tablet, a capsule, etc., with higher daily intakes or daily doses being suitable for administration so that some or all of the compounds for administration are available when taken orally as multiple oral dosage forms (of one type or of multiple types of oral dosages forms). Furthermore, it is noted that additional doses can be taken at different times of the day, in some instances, for a total daily dose.

It is also noted that when discussing the methylthioninium salt-containing compositions or any of the related methods herein, discussions of one type of example are considered applicable to other examples whether or not they are explicitly discussed in the context of that example unless expressly indicated otherwise. Thus, for example, when discussing caffeine in the context of one or more of the compositions described herein, such disclosure is also relevant to and directly supported in context of other composition examples as well as any of the methods described herein, and vice versa. Furthermore, for simplicity and illustrative purposes, numerous specific details are set forth in order to provide a thorough understanding of the present disclosure. It will be readily apparent however, that the present disclosure can be practiced without limitation to some of these specific details. In other instances, certain methods, systems, materials, and structures have not been described in detail so as not to obscure the present disclosure.

Furthermore, terms used herein will have their ordinary meaning in the relevant technical field unless specified otherwise. In some instances, there are terms defined more specifically throughout the specification, with a few more general terms included at the end of the specification. These more specifically defined terms have the meaning as described herein.

### Combination Treatments of Methylthioninium Salt and a Secondary Physiologically Active Compound Coupled with Physiological Therapy

A physiological protection or enhancement system can include a methylthioninium salt-containing composition and a physiological therapy device. The methylthioninium salt-containing composition can include a methylthioninium salt and a secondary physiologically active compound, such as urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or a combination thereof. The physiological therapy device can be used in combination with the methylthioninium salt-containing composition, and can include such physiological therapy devices as a thermal therapy device, an electromagnetic therapy device, an oxygenation therapy device, or a combination thereof.

In another example, a method of protecting or enhancing a physiological function can include orally co-administering a methylthioninium salt and a secondary physiologically active compound to a subject. The secondary physiologically active compound can include, for example, urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or a combination thereof. The method can also include subjecting the subject to physiological therapy such as thermal therapy, electromagnetic therapy, oxygenation therapy, or a combination thereof. The physiology therapy can be conducted while the subject is dosed with clinically effective blood serum levels of the methylthioninium salt and the secondary physiologically active compound. The term “clinically effective blood serum levels” refers to elevated blood serum levels of the methylthioninium salt and the secondary physiologically active compound which have a physiological effect when taken orally.

In other examples, a physiological enhancement system can include a liquid nutritional supplement composition including from about 80 wt % to about 99 wt % liquid carrier (or water); from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and from about 0.1 wt % to about 5 wt % methylated vitamin B12, from about 0.1 wt % to about 5 wt % vitamin C, or both. The system may further include a physiological therapy device for use in combination with the liquid nutritional supplement composition, such as a thermal therapy device, an electromagnetic therapy device, an oxygenation therapy device, or a combination thereof.

In other examples, a method of protecting or enhancing a physiological function can include orally administering a liquid nutritional supplement composition to a subject, the liquid nutritional supplement composition including from about 80 wt % to about 99 wt % liquid carrier (or water); from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and from about 0.1 wt % to about 5 wt % methylated vitamin B12, from about 0.1 wt % to about 5 wt % vitamin C, or both. The method can also include subjecting the subject to a physiological therapy selected from thermal therapy, electromagnetic therapy, oxygenation therapy, or a combination thereof while the subject is dosed with clinically effective levels of the liquid nutritional supplement composition to enhance the physiological function or enhance the absorption of methylthioninium salt and/or a secondary physiologically active compound.

With respect to the physiological protection and/or enhancement systems and the methods of protecting or enhancing the physiological function of a subject, it is noted that the thermal therapy can be in the form of cold plunge therapy, cryotherapy, sauna therapy, localized heat or cold therapy, e.g., simulating cold plunge with localized cold exposure such as to the face, or a combination thereof. The electromagnetic therapy can be in the form of red-light therapy, pulse electromagnetic field therapy, electric stimulation therapy, or a combination thereof. The oxygenation therapy can be in the form of hyperbaric oxygen therapy (HBOT), enhanced oxygen therapy, e.g., via an oxygen tank and mask or cannula, or a combination thereof. These various therapies can be conducted using various suitable devices, such as a hyperbaric oxygen chamber for HBOT, a red-light bed/mat or red-light lamps for delivering red-light therapy, etc. Regarding the methylthioninium salt, the secondary physiologically active compound, or the methylthioninium salt-containing composition (which is co-formulated with both compounds) can be presented for use as an oral dosage form, such as tablet(s), capsule(s), soft-gel(s), powder, liquid suspensions or solutions, transmucosal oral formulations, etc. In some examples, the methylthioninium salt can be in the form of methylthioninium chloride, which is often referred to commercially as methylene blue.

In accordance with some more specific examples of the present disclosure, multiple types of secondary physiologically active compounds can be combined with a methylthioninium salt (orally co-administered either separately or as a methylthioninium salt-containing composition) to provide various health benefits. MB1-MB14 are presented below in Table 1 by way of example to succinctly illustrate various combinations of secondary physiologically active compounds that can be co-formulated together with the methylthioninium salt or otherwise co-administered from separate compositions relative to the methylthioninium salt. Furthermore, additional combinations are also provided that include a liquid mineral blend of mineral blend of salts, oxides, and/or chlorides, for example, that can be used with the physiological therapies as described herein. These various compositions are provided in Table 1, as follows:

TABLE 1
Example Methylthioninium Salt-containing Compositions
and Liquid Nutritional Supplement Compositions

| Comp. ID | Ingredients |
|---|---|
| MB1 | MB^{†}, caffeine, L-theanine |
| MB2 | MB^{†}, green tea, guarana, L-theanine |
| MB3 | MB^{†}, mineral salts/oxides/chlorides, |
| | hydrogen peroxide (formed in situ) |
| MB4 | MB^{†}, mineral salts/oxides/chlorides, |
| | hydrogen peroxide, colloidal silver |
| MB5 | MB^{†}, vitamin C |
| MB6 | MB^{†}, glycine, N-acetylcysteine, vitamin B3 (niacinamide) |
| MB7 | MB^{†}, cannabidiol (CBD), vitamin B6, vitamin E, manganese |
| MB8 | MB^{†}, urolithin A |
| MB9 | MB^{†}, pyrroloquinoline quinone |
| MB10 | MB^{†}, tesofensine |
| MB11 | MB^{†}, nicotinamide adenine dinucleotide (NAD) |
| MB12 | MB^{†}, urolithin A, nicotinamide adenine dinucleotide (NAD) |
| MB13 | MB^{†}, urolithin A, pyrroloquinoline quinone |
| MB14 | MB^{†}, urolithin A, pyrroloquinoline quinone, tesofensine |
| LNS1 | Mineral salts/oxides/chlorides, methylated B12, vitamin C |
| LNS2 | Mineral salts/oxides/chlorides, methylated B12 |
| LNS3 | Mineral salts/oxides/chlorides, vitamin C |

MB^{†} refers to methylthioninium chloride in these examples.

In addition to the co-administration of these or other combinations of compounds, in many instances pairing the oral administration of these compounds or compositions with any of a number of physiological treatments can provide additional benefits. In accordance with this, the term “physiological treatment” refers to any treatment or therapy activity designed to relieve or reverse symptoms or conditions that interfere with the physiological functioning of the human body, or to enhance physiological functioning of the human body that may already be operating at an acceptable level. These physiological therapies can work to enhance the benefits of the oral delivery of the compounds or compositions described herein, and in some instances provide synergy with respect to certain physiological goals. Some more specific categories of physiological therapies and several more specific physiological therapies (PT1-PT9) from each category that can be used in connection with the present systems and methods are provided by way of example in Table 2, as follow:

TABLE 2
Example Physiological Therapies

| Therapy ID | Therapy Category | Physiological Therapy |
|---|---|---|
| PT1 | Thermal Therapy | Cold Plunge Therapy |
| PT2 | | Cryotherapy |
| PT3 | | Sauna Therapy |
| PT4 | | Localized Heat or Cold Therapy |
| PT5 | Electromagnetic Therapy | Red-light Therapy |
| PT6 | | Pulsed Electromagnetic |
| | | Field Therapy |
| PT7 | | Electric Stimulation Therapy |
| PT8 | Oxygenation Therapy | Hyperbaric Oxygen Therapy |
| PT9 | | Enhanced Oxygen Therapy |

Combining one or more of these types of physiological therapies and/or more specific physiological therapies listed in Table 2 with the oral delivery of methylene blue and one or more secondary physiologically active compound(s) as shown in Table 1 can produce various benefits to the human body. For example, these combinations can provide benefits related to the central nervous system (CNS), including benefits related to neural and overall brain function, e.g., enhanced memory or memory retention, cognition, psychiatric health (e.g., anxiety, depression, Alzheimer's disease, attention disorders, etc.), neuromodulation (e.g., production and regulation of dopamine, norepinephrine, serotonin, GABA, neuropeptides, etc., and/or receptor modulation). Other health benefits that may be realized include improved mitochondrial health (e.g., regulation and/or repair of mitochondria), reduction or control of inflammation, enhanced cellular health and/or protection (e.g., antioxidants for free radical), muscle health (e.g., strength, growth, repair, etc.), gut health, weight loss or management, metabolism glucagon-like peptide-1 (GLP-1) enhancement, and others.

Some specific examples of pairings of secondary physiologically active compounds and physiological therapies that can be implemented (in addition to the oral delivery of the methylthioninium salt) are provided as follows. For example, the methylthioninium salt can be co-administered with urolithin A, pyrroloquinoline quinone, guarana, L-theanine, caffeine, vitamin C, mineral blend of salts, oxides, and/or chlorides, or a combination thereof, and the physiological therapy can include the use of oxygenation therapy, e.g., a hyperbaric oxygen therapy (HBOT). In another example, the methylthioninium salt can be co-administered with urolithin A, pyrroloquinoline quinone, guarana, L-theanine, green tea, mineral blend of salts, oxides, and/or chlorides, N-acetylcysteine (NAC), vitamin B3, e.g., niacinamide and/or nicotinic acid, glycine, or a combination thereof, and the physiological therapy can include electromagnetic therapy, e.g., red-light therapy and/or pulse electromagnetic field therapy device. In another example, the methylthioninium salt can be co-administered with urolithin A, pyrroloquinoline quinone, green tea, guarana, vitamin C, or a combination thereof, and the physiological therapy can include thermal therapy, e.g., cold plunge therapy or targeted tissue cooling to partially simulate the effects of cold plunge therapy. Other combinations can also be effective for treatments resulting in physiological protection and/or enhancement of cellular health, tissue health, systemic health, etc.

### Primary Physiologically Active Compound

### Methylthioninium Salt, e.g., Methylthioninium Chloride (Methylene Blue)

Methylthioninium salts, such as methylthioninium chloride (or methylene blue), are synthetic chemical compounds with a crystalline structure. For example, methylthioninium salt has a specific crystalline structure appearing dark green as a powder at room temperature and blue when mixed in water. The blue color can be lost with time when at rest, e.g., in its reduced state due to lack of interaction with oxygen), but re-emerges as blue when agitated, e.g., in its oxidized state. Because of these unique properties, it has been used diagnostically to distinguish tissues and fluids in a lab setting. Methylthioninium salt can also function similarly to hemoglobin, the substance responsible for transporting oxygen to the body's tissues and organs, making it a good candidate for various therapies, though its use orally over the years has led to different levels of success. In further detail, methylthioninium salt is a formal derivative of phenothiazine, with the hydrated form having three (3) molecules of water per single molecule of the methylthioninium salt compound. The structure of methylthioninium chloride is shown by way of example in Formula I, as follows:

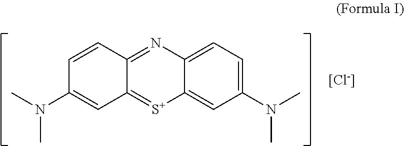

In accordance with the present disclosure, when a methylthioninium salt such as methylthioninium chloride is co-formulated with the secondary physiologically active compound, the impact this combination can have on the protection and/or enhancement of various physiological functions can be particularly beneficial. For example, methylthioninium salt can be delivered to provide improved mitochondrial function. Methylthioninium chloride, for example, is a hormetic substance, meaning that it can be more effective at relatively low dosages. In accordance with this, the methylthioninium salt can be present in a composition for delivery as part of a single daily dosage form or a divided daily dosage form, e.g., multiple oral dosage forms (2 tablets, 1 tablet and one liquid suspension shot, etc.) taken at the same time or separated by time during a single 24 hour period). Regardless of how the total daily dosage is orally delivered, the methylthioninium salt can be delivered daily at from about 1 mg to about 50 mg, from about 1 mg to about 40 mg, from about 1 mg to about 30 mg, from about 1 mg to about 25 mg, from about 1 mg to about 20 mg, from about 1 mg to about 15 mg, from about 1 mg to about 12 mg, from about 2 mg to about 50 mg, from about 2 mg to about 40 mg, from about 2 mg to about 30 mg, from about 2 mg to about 25 mg, from about 2 mg to about 20 mg, from about 2 mg to about 15 mg, from about 2 mg to about 12 mg, from about 4 mg to about 20 mg, or from about 4 mg to about 12 mg.

Regarding the secondary active physiologically active compound that is co-delivered on a daily basis, the daily dosage (in milligrams) for oral delivery of the secondary physiologically active compound (or compounds) can depend on the specific compound(s) selected and the physiological effect to be targeted. Example weight ratios are provided hereinafter as it relates to the oral co-administration of the methylthioninium salt and the secondary physiologically active compound(s). Notably, in some instances, the multiple compounds can be orally co-administered, which can provide synergy in treating various conditions, including any of a number of cognitive and/or neurological conditions, and others. Furthermore, further physiological protection and/or enhancement can be achieved through the use of one or more of the physiological therapies also described herein.

It is generally understood that mitochondria are the energy producers of the cells, and often neurodegenerative and psychiatric disorders can lead to mitochondrial damage. The neuroprotective properties of methylthioninium salts can, for example, improve mitochondrial function such that a subject may see improvements in memory retention, cytochrome oxidase activity, ATP generation, etc., thus assisting with the maintenance of neural health and cognitive function. Furthermore, mitochondrial disfunctions can decrease energy transfer to the cells resulting in cellular damage and/or death, e.g., cellular damage can increase neuroinflammation. Furthermore, when mitochondria disfunctions, energy transfers may cease to occur, leading to cellular death. Additionally, toxic build up can occur, leading to oxidative stress and the release of free radicals (and ultimately, loss of neurons). Methylthioninium salt, such as methylene blue, can provide some alternative energy transfer that is effective for protecting neurons by increasing ATP production (which provides energy for the brain via mitochondria respiration). More specifically, as there is a link between methylthioninium salt and neurodegenerative disorders and/or energy metabolism, oral delivery of methylthioninium salt can result in electrons being received from nicotinamide adenine dinucleotide (NADH and/or NAD+, but referred to generally as “NAD”) in the presence of Complex I and donate them to Cytochrome C, thus providing an alternative electron transfer pathway. This can result in an increase in oxygen consumption, a decrease in glycolysis, and an increase in glucose uptake enhancing cerebral blood flow. This can be particularly useful for therapeutic uses that increase ATP production after passing through the blood-brain barrier.

In further detail, methylthioninium salt, particularly when dosed with the second physiologically active compound that impacts the mitochondria, can act to enhance not only the function of mitochondria, but can act to repair mitochondrial damage and/or provide conditions for mitophagy to occur to degrade damaged mitochondria by autophagy. Examples of such secondary physiologically active compounds that may provide this secondary mitochondrial benefit include urolithin A, pyrroloquinoline quinone, tesofensine, etc. Furthermore, methylthioninium salt can provide benefits as a superoxide (antioxidant) to reduce or eliminate free radicals that assist in repairing impairments in mitochondrial function and cellular metabolism. In connection with this, by improving mitochondrial function and reducing oxidative damage, methylthioninium salt can contribute to anti-aging in some subjects. For example, methylthioninium salt can bypass Complex I/III activity in the mitochondria, reducing oxidative stress and improving mitochondrial efficiency, which can be beneficial for treating age-related conditions, such as neurodegeneration, memory loss, skin aging, and the like.

In other examples, when the methylthioninium salt is admixed with various other secondary physiologically active compounds, such as caffeine, L-theanine, green tea, guarana, magnesium, vitamin C, amino acid, e.g., glycine, N-acetylcysteine (NAC), vitamin B3, e.g., niacinamide and/or nicotinic acid, black pepper, vitamin B6, manganese, cannabinoids, nicotine, and/or others, the efficacy of the methylthioninium salt can be enhanced when combined at appropriate weight ratios or daily dosages. In some instances, the multiple compounds can exhibit synergy in treating various conditions, including any of a number of cognitive and/or neurological conditions.

In further detail, methylthioninium salt, particularly when dosed with some of the other secondary physiologically active compounds described herein, can act to enhance the depletion of dopamine common in many central nervous system conditions, such as Parkinson's disease. Furthermore, methylthioninium salt can provide the benefits of enhancing neuroprotection within the central nervous system acting as a superoxide (antioxidant) to reduce or eliminate free radicals, reducing cerebral ischemia reperfusion damage in a transient focal cerebral ischemia model, reducing formation of amyloid plaques and neurofibrillary tangles, and repairing impairments in mitochondrial function and cellular metabolism, e.g., reducing plaque formation, etc. In short, as the brain uses a disproportionate amount of oxygen and sugar (ATP) compared to its size, orally treating a subject with a condition associated with the central nervous system can provide some of the raw materials used for proper brain function.

### Secondary Physiologically Active Compounds

### Urolithin A

Urolithin A is a postbiotic compound that is instrumental in triggering mitophagy, which is a specific type of autophagy related to cellular recycling. More specifically, urolithin A is a benzo-coumarin (or dibenzo-α-pyrones) compound and is a metabolite of ellagitannin precursors, which can be transformed to urolithin A by certain gut bacteria. In addition to the ellagitannin precursors, Urolithin A has other precursors, such as ellagic acids. More specifically, ellagitannins can be hydrolyzed in the gut to release ellagic acid, which can be further processed by the gut microflora into urolithin A, e.g., via removal of lactone and hydroxyl groups. These precursors are prevalent in various foods and editable plants, such as nuts, e.g., walnuts, pomegranates, berries, e.g., strawberries, raspberries, blackberries, cloudberries, etc., teas, muscadine grapes, and many others.

Even though the precursors are found in natural foods and can be consumed readily, the metabolite urolithin A is not found typically in food sources per se. Furthermore, there are some humans, in particular, that do not efficiently convert ellagitannins into urolithin A, and other humans that do not convert ellagitannins into urolithin A at all. For example, the transformation of ellagic acids into urolithin A depends greatly on the microflora composition in the gut of the subject, which can vary significantly. As fewer and fewer natural foods are being consumed in recent years, the microflora composition of the gut of many individuals may not be optimally established for this transformation. A change in diet may provide an improved microbiome, but genetics may also play a role in the body's ability to make this transformation from precursors to achieving blood serum levels of urolithin A that would be effective in promoting overall mitochondrial health via a healthy level of mitophagy.

In accordance with this, it is believed that less than about 40%-50% of humans can effectively generate urolithin A from its precursors in situ. Thus, for such individuals, supplementation of urolithin A can be highly beneficial, particularly when co-formulated with a methylthioninium salt as described herein, as both together provide a highly effective composition for enhancing the impact that the mitochondria has on the body at a cellular level.

Urolithin A can enter systemic circulation via absorption through the intestines, making it an acceptable compound for oral delivery when dosed at appropriate levels. Urolithin A, as an intact molecule, is shown in Formula II below.

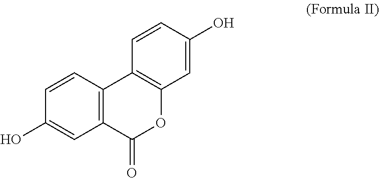 [SMILES: O=c1oc2cc(O)ccc2c2ccc(O)cc12]

Furthermore, when urolithin A is within the body, e.g., the gut, intestinal absorption cells (or enterocytes), systemic circulation, tissue, organs, (hepatocytes such as in the liver), etc., various types of compound transformations may occur in situ, e.g., glucuronidation, methylation, sulfation, etc. As a result, urolithin A has various derivatives, including urolithin A glucuronide, urolithin A sulfate, etc., which can be present in systemic circulation prior to being excreted via the urine. In accordance with this, reference to “urolithin A” herein includes the form of urolithin A shown at Formula II, as well as its salts and derivatives that may be beneficial within the body. Typically, urolithin A as shown in Formula II is delivered orally as described herein, but could be delivered as a salt or as a derivative in some instances.

When co-formulating or orally co-administering with the methylthioninium salt, the methylthioninium salt to urolithin A weight ratio can be from about 1:600 to about 1:4, from about 1:300 to about 1:4, from about 1:200 to about 1:5, from about 1:200 to about 1:6, from about 1:150 to about 1:10, from about 1:100 to about 1:20, or from about 1:80 to about 1:25, either co-formulated or administered as a daily dose (whether by using one oral dosage form, e.g., 1 tablet, or multiple dosage forms, e.g., 2-4 tablets). In terms of daily dosages of the urolithin A with the methylthioninium salt, such dosages can range from about 100 mg to about 1200 mg, from about 200 mg to about 1200 mg, from about 250 mg to about 1000 mg, from about 300 mg to about 750 mg, from about 400 mg to about 900 mg, from about 500 mg to about 750 mg, or from about 200 mg to about 500 mg, for example, e.g., as a single tablet or divided over multiple tablets. For example, if a daily dose to be given to a subject is about 300 mg urolithin A divided over two tablets, each tablet may contain about 150 mg. On the other hand, the 300 mg of urolithin A could be given as a daily dose from a single dosage form, e.g., a single tablet.

As humans age and/or are exposed to various environmental toxins and/or stresses, e.g., EMF, chemical, psychological stress, mitochondrial function tends to decline, leading to increases in chronic disease (due to progressive failure of the mitochondria). In addition to chronic disease, these factors can also lead to reduced energy, slower metabolism, cognitive decline, etc. Repair of the mitochondria, e.g., with mitochondrial DNA damage such as by processes related to identification of poor cellular function, can occur and when repair is warranted, mitophagy or autophagy may be initiated via cellular reduction along with cellular replacement.

With these factors and others negatively impacting the mitochondria, it has been found that urolithin A provides many benefits, particularly when co-administered in a composition with methylthioninium salts. For example, urolithin A can: i) stimulate the renewal of mitochondria by encouraging replacement of damaged or dysfunctional mitochondria with higher functioning mitochondria; ii) stimulate anti-inflammatory properties and assist with the reducing of inflammation markers associated with chronic disease often exacerbated by inflammation; iii) provide a potent antioxidant to protect cells from damage caused by free radicals; iv) improve muscle strength, growth, and muscle repair (at a cellular level) by influencing health markers and muscle genes associated with mitochondria present in the muscle tissue; and v) positively impact gut health by altering the gut microbiota, improving alpha diversity, restoring colonic barrier function, etc.

To provide one example of the benefits of urolithin on mitochondrial health, the health of muscle tissue can be considered. In accordance with this, the administration of urolithin A can be particularly useful in middle-aged and elderly individuals for muscle health, and furthermore, is sufficiently bioavailable and well tolerated as an oral supplement. Examples of the benefits urolithin A has on muscle tissue include the maintaining or enhancing of muscle endurance, strength, exercise performance, biomarkers of mitochondrial health, and/or overall muscle health. Furthermore, urolithin A can also lower levels of plasma acylcarnitine compounds and C-reactive proteins, which provide evidence for enhanced mitochondrial efficiency and reduced inflammation.

In accordance with this, it has been recognized as described herein that the combination of Urolithin A and a methylthioninium salt, such as methylene blue, can provide high levels of support for mitochondrial health and muscle function. For example, as mentioned, urolithin A can play a role in promoting mitochondrial mitophagy and can be complemented by the antioxidant properties of methylthioninium salts leading to improved cellular energy production and reduced oxidative stress. As an example, this combination can be particularly beneficial for enhancing physical performance and overall metabolic health when taken at appropriate dosages as described herein.

### Pyrroloquinoline Quinone (PQQ)

Pyrroloquinoline quinone is a compound that is related to the B-vitamin family, which provides strong antioxidant support for mitochondria, which can support cellular energy and assist with maintaining a healthy mind, including supporting a healthy memory. For example, pyrroloquinoline quinone can enhance or support mitochondrial general health as well as growth, and furthermore is neuroprotective against mental function that may be lost due to aging or other conditions. In addition to being effective for mitochondrial health as it relates to mental function, it can also support muscle and other tissue maintenance and growth. Regarding its antioxidant properties, pyrroloquinoline quinone has been reported as being more than two orders of magnitude (>100×) more effective at triggering continuous cycling compared to more common antioxidants, such as vitamin C and various phenolic compounds. In other words, pyrroloquinoline quinone can cycle back between multiple forms, including between a first active form and a second antioxidant form. Cycling back and forth between these two forms, depending on conditions, can provide the ability for performing antioxidant functions as it cycles back and forth for repeated oxidation and reduction reactions. This cycling for repeated antioxidant use provides an excellent way of supporting the central nervous system, e.g., brain, spinal cord, and other nervous system cells or tissue.

In further detail, it is noted that pyrroloquinoline quinone can be administered and/or co-formulated with methylthioninium salts, such as methylene blue, providing a potent antioxidant that supports mitochondrial biogenesis and function. Furthermore, pyrroloquinoline quinone can enhance nicotinamide adenine dinucleotide (NAD) production and use, assisting with mitochondrial efficiency. In further detail, pyrroloquinoline quinone can enhance the housekeeping of the cell with its function supporting mitochondrial mitophagy while the methylthioninium salt can enhance ATP production. As ATP is the source of cellular energy storage and delivery produced by the mitochondria, health implications as it relates to the heart, brain, muscles, and other major organs can benefit greatly from the presence of pyrroloquinoline quinone, particularly when there is some dysfunction, such as the slowing of this functionality as a result of aging. The presence of pyrroloquinoline quinone can assist with supporting the appropriate structure and/or function of the mitochondria.

The structure of pyrroloquinoline quinone is shown by way of example in Formula III, as follows:

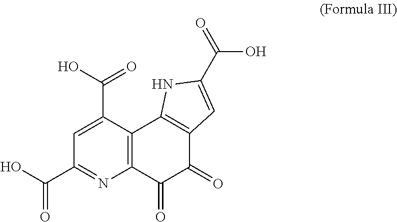 [SMILES: O=C(O)c1cc(C(=O)O)c2c(n1)C(=O)C(=O)c1cc(C(=O)O)[nH]c1-2]

When co-formulating or orally co-administering with the methylthioninium salt, the methylthioninium salt to pyrroloquinoline quinone weight ratio can be from about 1:20 to about 50:1, from about 1:20 to about 30:1, from about 1:10 to about 40:1, from about 1:5 to about 25:1, or from about 1:5 to about 20:1, for example. These weight ratios can be administered orally as a single composition or in separate dosage forms. In terms of individual dosage forms of the pyrroloquinoline quinone with the methylthioninium salt, such dosages of the pyrroloquinoline quinone can range from about 0.25 mg to about 20 mg, from about 1 mg to about 15 mg, from about 0.5 mg to about 10 mg, from about 0.5 mg to about 4 mg, from about 0.75 mg to about 3 mg, or from about 1 mg to about 2.5 mg. Regarding daily dosages to be administered with the methylthioninium salt, the pyrroloquinoline quinone can be administered daily (as a single dose or multiple divided doses) at from about 0.5 mg to about 40 mg, from about 1 mg to about 40 mg, from about 1 mg to about 30 mg, from about 1 mg to about 20 mg, from about 1 mg to about 8 mg, from about 1.5 mg to about 6 mg, or from about 2 mg to about 5 mg as a single dosage form, e.g., tablet, or divided over multiple dosage forms. For example, if a daily dose to be given to a subject includes about 10 mg pyrroloquinoline quinone divided over two tablets, each tablet may contain about 5 mg.

### Tesofensine

Tesofensine is a serotonin-norepinephrine-dopamine reuptake inhibitor (SNDRI). More specifically, tesofensine functions as a triple monoamine reuptake inhibitor, targeting the dopamine transporter (DAT), norepinephrine transporter (NET), and serotonin transporter (SERT). By inhibiting these transporters, tesofensine can increase the levels of dopamine, norepinephrine, and/or serotonin in the brain. This functionality can assist with curbing appetite and enhancing feelings of fullness and satisfaction, even after eating smaller than usual portions of food. Due to this, tesofensine was primarily developed for the treatment of obesity. For example, tesofensine trials have indicated clinical benefits for weight loss, inhibiting the reuptake of neurotransmitters such as serotonin, norepinephrine, and dopamine, which play biological roles in appetite control and metabolism.

In addition to appetite suppression and treatment of obesity, tesofensine may also enhance cognitive function as a result of its impact on the DAT, NET, and SERT. Related to cognitive enhancement is its impact on modulating neuronal activity in the lateral hypothalamus, silencing GABAergic neurons that are involved in hunger signaling. In further detail regarding the impact that tesofensine has on cognition, this compound can indirectly potentiate cholinergic neurotransmission, which has beneficial effects on cognition, particularly in learning and memory in particular. Furthermore, tesofensine can improve mood and reduce anxiety in subjects with depression and anxiety disorders. There may also be a connection between cognitive enhancement and the treatment of obesity, as it can be effective more specifically in treating anxiety and depression in subjects with comorbid depression and anxiety. Tesofensine can also inhibit dopamine transporter proteins, allowing dopamine to stimulate receptors for a more extended period of time. This can provide longer lasting cognitive benefits, and in connection with weight loss, it can enhance the reward system of the brain, providing biological activity causing subjects to feel more satisfied with smaller meals and a reduced desire to snack. In additional detail, tesofensine may provide positive results when treating sexual dysfunction, ADHD, alcohol addiction, sleep disorders, low energy, etc.

The structure of tesofensine is shown by way of example in Formula IV below.

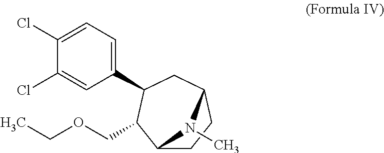 [SMILES: CCOC[C@@H]1[C@@H](c2ccc(Cl)c(Cl)c2)C[C@@H]2CC[C@H]1N2C]

In accordance with the present disclosure, it has been found that co-administering or co-formulating tesofensine with methylthioninium salts, such as methylene blue, can also promote overall mitochondrial health. Methylthioninium salts such as methylene blue, for example, can provide the mitochondrial benefits described previously. Tesofensine, on the other hand, can improve metabolic markers related to insulin sensitivity and blood lipid levels, providing a good option for subjects looking to improve their overall metabolic health. When combined, methylthioninium salt and tesofensine can offer synergistic benefits in both cognitive and metabolic health. More specifically, tesofensine has the ability to enhance neurotransmitter levels, which can be complimented by the neuroprotective properties of the methylthioninium salt, both leading to improved cognitive function and mood stabilization. Furthermore, the cardiovascular benefits of methylene blue, as an example, can further support the metabolic improvements induced by tesofensine as described above. More specifically, methylene blue as an example can provide neuroprotective and cognitive-enhancing properties to a subject, improving mitochondrial efficiency. This can result in heightened energy, better physical well-being, and improved mental capabilities. Thus, when combined with tesofensine, the methylthioninium salt can actually amplify the cognitive benefits by further enhancing mitochondrial function and reducing oxidative stress.

In some examples, the combination of a methylthioninium salt with tesofensine can be further supplemented by one or more of the other compounds described herein, including urolithin A, pyrroloquinoline quinone, and/or nicotinamide adenine dinucleotide (NAD). In these examples, there may be a synergistic benefit to combining pyrroloquinoline quinone with compositions containing the methylthioninium salt and the tesofensine. As mentioned, pyrroloquinoline quinone is a potent antioxidant that supports mitochondrial biogenesis and function. Combining pyrroloquinoline quinone with tesofensine can provide enhancement in energy metabolism, which may also lead to weight loss. Pyrroloquinoline quinone's neuroprotective effects can also complement the administration of the tesofensine as it relates to cognitive benefits. In other examples, there may be a synergistic benefit to combining urolithin A with compositions containing the methylthioninium salt and the tesofensine. As mentioned, urolithin A can improve mitochondrial health and muscle function, promoting mitophagy (the process of removing damaged mitochondria), thereby enhancing cellular energy production and overall metabolic health. When combined with tesofensine, Urolithin A can act to enhance overall energy levels and physical performance, which is particularly useful for individuals engaging in physical activity as part of their weight loss regimen.

Thus, as tesofensine offers a range of health benefits, particularly related to weight loss and cognitive enhancement, by modulating key neurotransmitters in the brain, when combined with compounds like methylthioninium salts, pyrroloquinoline quinone, and/or Urolithin A, these benefits can be further amplified. For example, such combinations can offer a comprehensive approach to improving both metabolic and cognitive health. The choice of combination should be tailored to individual health goals and specific therapeutic needs, providing a personalized strategy for enhanced overall well-being. By understanding the mode of action and potential benefits of these combinations, healthcare providers can better tailor treatments to meet the specific needs of their patients.

When co-formulating or orally co-administering with the methylthioninium salt, the methylthioninium salt to tesofensine weight ratio can be from about 1:2 to about 100:1, from about 1:1 to about 75:1, from about 2:1 to about 50:1, from about 4:1 to about 40:1, from about 5:1 to about 30:1, or from about 6:1 to about 20:1, for example. These weight ratios can be administered orally as a single composition or in separate dosage forms. In terms of individual dosage forms including the tesofensine and the methylthioninium salt, such dosages of the tesofensine can range from about 0.125 mg to about 2 mg, from about 0.25 mg to about 1.75 mg, from about 0.4 mg to about 1.75 mg, from about 0.5 mg to about 1.5 mg, or from about 0.5 mg to about 1 mg. Regarding daily doses (single or divided daily dosages) to be administered with the methylthioninium salt, the tesofensine can be administered daily at from about 0.25 mg to about 4 mg, from about 0.5 mg to about 4 mg, from about 0.5 mg to about 3.5 mg, from about 0.8 mg to about 3.5 mg, from about 1 mg to about 3 mg, or from about 1 mg to about 2 mg. For example, if a single daily dose to be given to a subject is about 2 mg divided over two tablets, each tablet may contain about 1 mg. On the other hand, the 2 mg daily dose of tesofensine could be given as a daily dose from a single dosage form, e.g., a single tablet.

### Nicotinamide Adenine Dinucleotide

In accordance with examples of the present disclosure, the co-administration or co-formulation of a methylthioninium salt with one or more secondary compounds such as urolithin A, pyrroloquinoline quinone, and/or tesofensine can further be enhanced by adding a tertiary compound, such as nicotinamide adenine dinucleotide (NAD+ and/or NADH, but referred to generally as NAD). NAD is a versatile compound when present within the body of a subject, and thus, the co-administration or co-formulation of a methylthioninium salt and one of these secondary compounds can provide further cognitive and/or metabolic benefits. For example, NAD can provide the raw materials for establishing a pathway for the methylthioninium salt to receive electrons from nicotinamide adenine dinucleotide. In some examples when pyrroloquinoline quinone is included as a secondary compound, it can also assist the methylthioninium salt in this regard. To illustrate, as there is a link between methylthioninium salts and energy metabolism, an enhanced functionality of this compound in the body can likewise improve energy. For example, the NAD can provide an alternative electron transfer pathway which may result in an increase in oxygen consumption, a decrease in glycolysis, and an increase in glucose uptake. In some examples, this combination can be particularly useful for producing energy suitable for brain function, as the ATP production enhanced from this combination can occur after passing through the blood-brain barrier.

If NAD is included in the composition or co-administered with the methylthioninium salt (and other secondary physiological active compounds in some examples), example methylthioninium salt to nicotinamide adenine dinucleotide weight ratios can be from about 1:100 to about 5:2, from about 1:75 to about 2:1, from about 1:50 to about 3:2, from about 1:50 to about 1:1, or from about 1:25 to about 5:2. Regarding the daily dosage(s) of the nicotinamide adenine dinucleotide to be co-administered with the methylthioninium salt, example daily dosage ranges can be from about 20 mg to about 200 mg, from about 20 mg to about 120 mg, from about 20 mg to about 100 mg, from about 30 mg to about 100 mg, or from about 40 mg to about 100 mg. If divided over multiple oral dosage forms to be taken together or at different times of the day, e.g., 2 tablets, each tablet may include, for example, from about 10 mg to about 100 mg, from about 10 mg to about 75 mg, from about 7.5 mg to about 60 mg, from about 10 mg to about 50 mg, or from about 20 mg to about 50 mg.

### Caffeine

Caffeine is a compound that can be used to stimulate the central nervous system (CNS), and can also increase the production of dopamine, which is a neurochemical heavily involved in controlling concentration and focus. In additional detail, caffeine is chemically similar to nucleoside adenosine, which is why it can interact with the brain. Thus, caffeine can increase dopamine production and can block adenosine receptor sites. Furthermore, caffeine can constrict blood vessels and reduce blood flow. The chemical structure of caffeine is shown by way of example in Formula II, as follows:

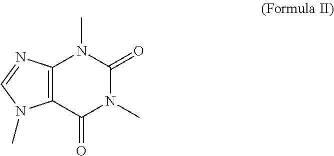

In further detail, caffeine can provide the following benefits, if dosed correctly, namely provide energy as a stimulant, boost brain health and mood due to the presence of antioxidants, improve focus and concentration, etc. Furthermore, coadministration of caffeine with L-theanine (described in greater detail below) provides a psychoactive synergistic effect promoting improved attention and alertness. Regarding dosing, the caffeine can be included in any of the methylthioninium salt-containing compositions described herein in a single dose at from about 40 mg to about 225 mg, from about 50 mg to about 200 mg, or from about 75 mg to about 150 mg, for example.

The caffeine can be added in a purified or substantially purified form, e.g., as an extract, and/or can be added via introduction with other compounds that naturally include some caffeine. Examples include green tea or guarana, both of which include a considerable concentration of caffeine along with other compounds. For example, green tea extract may contain more caffeine than green tea catechins in some instances. Green tea extract or green tea powder may include from about 15 wt % to about 20 wt % caffeine, though that depends on the extract, powder, dilution, etc. However, the benefit of using green tea as a caffeine source can be the presence of the green tea catechins, e.g., epigallocatechin (EGCG), epicatechin-3-gallate (ECG), epigallocatechin (EGC), epicatechin (EC), etc. Green tea can be included in the methylthioninium salt-containing compositions at from about 20 mg to about 300 mg, from about 20 mg to about 200 mg, or from about 20 mg to about 120 mg. The green tea catechins can be present in a single dose of the methylthioninium salt-containing compositions at from about 1 mg to about 100 mg, from about 2 mg to about 75 mg, or from about 3 mg to about 50 mg, for example.

The caffeine content provided by the green tea can be similar to that described previously unless there is a second caffeine source, e.g., guarana, in which case the total content of all caffeine sources would be calculated to range from about 40 mg to about 225 mg of caffeine content.

Guarana may be included as the sole caffeine source, or can be combined with other caffeine sources, such as green tea, purified or substantially purified caffeine, e.g. an extract, etc. Guarana is a dietary supplement or herb that is also a stimulant, and includes a considerable concentration of caffeine, e.g., about twice the content of caffeine as found in a typical coffee bean, e.g., about 2 wt % to about 8 wt % in the guarana seed. If guarana is included, it can be present at from about 50 mg to about 400 mg, from about 50 mg to about 220 mg, or from about 75 mg to about 200 mg, for example.

### L-Theanine

L-theanine is an amino acid found in nature that is not naturally produced by the body, but found in herbs and vegetables, specifically, green tea leaves, black tea leaves, and some mushrooms. As mentioned, L-theanine can be included in the methylthioninium salt-containing compositions of the present disclosure. Its presence can be even more effective when included in combination with caffeine (or a caffeine source), though formulations without both being present can also be effective for treating various conditions. L-theanine is shown by way of example in Formula III, as follows:

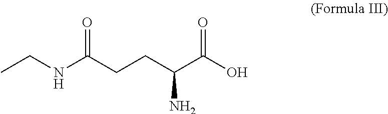

Supplementing with L-theanine can provide various benefits, including improved cognitive function, alleviation of psychological stress, maintenance of normal sleep/restful sleep, reduction of menstrual discomfort, enhanced mood, brain boosting effect, calming effect, etc. Similar to methylthioninium chloride, L-theanine can also cross the blood brain barrier, which can provide cognitive benefits and treat various disorders associated with the brain. In some examples, L-theanine can be present in a single dose of the methylthioninium salt-containing composition at from about 50 mg to about 450 mg, from about 75 mg to about 300 mg, or from about 75 mg to about 200 mg.

### Mineral Blends of Salts/Oxides/Chlorides (and Hydrogen Peroxide)

The mineral blend of salts, oxides, and/or chlorides added to some of the methylthioninium salt-containing compositions of the present disclosure can include a variety of minerals in oxide and/or chloride form. One example is magnesium chloride, which has good bioavailability in the body. Other examples include potassium chlorate, sodium chloride, and magnesium oxide. The mineral salts/oxides/chlorides (including chlorates) can be added, for example at from about 0.5 vol % to about 5 vol %, from about 0.5 vol % to about 3.5 vol %, from about 1 vol % to about 2.5 vol %, or at about 1.5 vol %. This liquid suspension can be orally administered as a liquid, or can be placed in one or more soft gel capsules.

When formulated in an aqueous suspension, for example, the presence of the mineral blend of salts, oxides, and/or chlorides in combination with water and the other components can result in liberating or forming hydrogen peroxide, which can also promote several health benefits. Example health benefits include bringing additional oxygen into the body. Most diseases proliferate when there is inadequate oxygen in the body, so the presence of hydrogen peroxide being formed in an aqueous liquid suspension can likewise have healing properties related to reduction of pain, detoxification of the body, and eliminating infection, for example.

### Colloidal Silver

Colloidal silver in the form of small elemental particles or “colloids” at relatively low doses can be an effective antimicrobial and can also promote healing properties, e.g., wound healing, gut health, etc., and can support the immune system. In some examples, colloidal silver used herein can be sized in the nano-scale, e.g., up to about 50 nm or so, at a relatively low concentration, up to about 100 ppm in a liquid suspension. Sizes and concentrations above these levels can be used as well in some instances, provided issues related to bioaccumulation can be addressed. In some examples, Ag404 colloidal silver from American Biotech Labs has been shown effective for oral delivery, with an average particle size from about 5 nm to about 15 nm in size. The bioactivity of Ag404 in particular may be related at least in part to its surface area to volume ratio due to its considerably small size, without substantial bioaccumulation. Whether this colloidal silver is used or another, a benefit to using colloidal silver can relate to its ability to steal electrons from pathogens and reduce the bio load of various pathogens in the body.

### Vitamin C

Vitamin C, which can be in the form of ascorbic acid and/or an ascorbate) is a water-soluble vitamin that is found in many fruits, and is considered an essential nutrient involved in tissue repair, formation of collagen, and the enzymatic production of some neurotransmitters as part of the central nervous systems. Vitamin C is also used for the functioning of several enzymes associated with the immune system. The structure of vitamin C (as ascorbic acid) is shown in Formula IV, as follows:

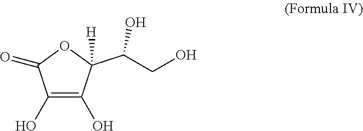 [SMILES: O=C1O[C@@H]([C@H](O)CO)C(O)=C1O]

In accordance with examples herein, Vitamin C can be particularly useful when combined with methylthioninium chloride in treating disorders and diseases associated with dementia due to its enzymatic activity related to neurotransmitters. For example, vitamin C is a potent antioxidant that can help protect mitochondria from oxidative stress. Mitochondria are particularly vulnerable to oxidative damage due to the high levels of reactive oxygen species (ROS) they generate during energy production. By scavenging ROS, vitamin C helps prevent damage to mitochondrial DNA, proteins, and lipids, thus supporting their function and overall health. As methylthioninium chloride also interacts with mitochondria, e.g., it can act as an electron carrier and help with ATP production, it can work well when combined with vitamin C. When used together, vitamin C and methylthioninium chloride can provide complimentary or even synergistic effects on mitochondria health. For example, both compounds have antioxidant properties, so when used together, they may provide enhanced protection against oxidative damage to mitochondria.

### Glycine

Glycine is the simplest of all of the stable amino acids, having a single hydrogen as its “R” group (present at the carboxyl group) rather than having a longer side chain. Glycine can act as an inhibitory neurotransmitter in the right circumstances, such as in the central nervous system, e.g., spinal cord, brainstem, and retina. When glycine receptors are activated, chloride can enter the neuro via ionotropic receptors, causing inhibitory postsynaptic potential. Furthermore, glycine is a co-agonist along with glutamate for NMDA receptors. Glycine plays multiple roles in the central nervous system, but notably, when dosed properly with other compounds of the methylthioninium salt-containing compositions of the present disclosure, it can also enhance the effectiveness of certain treatments. For example, glycine can also have neuroprotective properties and can assist with improving cognition, and can also assist with the prevention of the formation of beta-amyloid plaques. The structure of glycine is shown in Formula V, as follows:

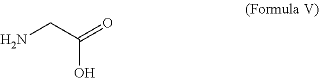

### N-Acetylcysteine (NAC)

N-acetylcysteine has many health benefits, including replenishing antioxidants, e.g., glutathione, brain health, ameliorating chronic respiratory conditions, enhancing fertility, etc.

As glutathione is one of the more bioactive antioxidants in the body, sufficient serum levels provide a good basis for neutralizing free radicals that can damage cells and tissue. With respect to central nervous system support, N-acetylcysteine can regulate glutamate levels, which is a valued neurotransmitter in the body which should be regulated within a safe range, e.g., not too much and not too little. Mental disorders, such as bipolar disorder, schizophrenia, obsessive-compulsive disorder (OCD), and substance use disorder are all conditions that can be tied to improper regulation of glutamate and/or glutathione. By replenishing glutathione and regulating glutamate in the central nervous system, brain health can be boosted. As a broad range of cognitive and behavioral systems rely on the neurotransmitter glutamate, proper serum levels of N-acetylcysteine in the body can promote cognition and behavior. In short, N-acetylcysteine is an antioxidant (and promotes the production of glutathione, which is also a powerful antioxidant) effective at protecting the central nervous system, and promoting improved cognition, memory, etc., particularly with subjects suffering from one form or another of dementia. The structure of N-acetylcysteine is shown in Formula VI, as follows:

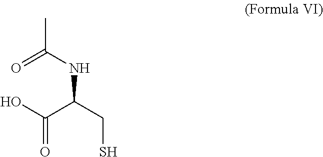

### Vitamin B

Regarding the B-complex vitamin content, any of a number of B-complex vitamins can be included in the various methylthioninium salt-containing compositions described herein. For example, the B-complex vitamins can be sourced from any of a number of liquid solutions or suspensions and/or dry B-complex vitamin sources. Example B-complex vitamins that can be included are thiamin (B1), riboflavin (B2), niacin or niacinamide (B3), pyridoxine (B6), folic acid or folate (B9), and/or cobalamin (B12). In some examples, the B-complex vitamins can be selected from vitamin B3 and/or vitamin B6. The vitamin B3, in some examples, can be in the form of niacinamide. In other examples, the vitamin B6 can be in the form of pyridoxal 5′ phosphate (PLP). In still other examples, the B-complex vitamin(s), where applicable, can be in a methylated form, which can assist with various metabolic processes. For example, methylated vitamin B9 and methylated vitamin B12 can be more bioavailable and useful when dosed appropriately, particularly with some subjects with gene mutations rendering their body inefficient for in situ methylation.

Regarding B-complex vitamins and their interaction with methylthioninium chloride, it is noted that administration of methylthioninium chloride can have a positive impact on neurodegenerative disorders and energy metabolism. With more specific reference to nicotinamide (or vitamin B3), it is noted that niacinamide can be converted to nicotinamide adenine dinucleotide (NAD, NADH), which is a versatile compound in the body. The coadministration of methylthioninium chloride with B-complex vitamins provides raw materials for establishing a pathway for the methylthioninium chloride to receive electrons from nicotinamide adenine dinucleotide (NADH) in the presence of Complex I and donating them to Cytochrome C. This provides an alternative electron transfer pathway causing an increase in oxygen consumption, a decrease in glycolysis and an increase in glucose uptake enhancing cerebral blood flow.

This mechanism can provide protection to neurons associated with neurodegenerative diseases by activating AMP-activated protein kinase inhibiting harmful enzymes downstream. Additionally, this can also enhance mitochondrial oxidative phosphorylation via alternative mitochondrial electron transfer, offering protection against neurodegenerative diseases. In further detail, it is understood that methylthioninium chloride crosses the brain-blood barrier easily to start ATP production. ATP provides energy for the brain through mitochondria respiration as an alternative electron carrier that transfers electrons without affecting mitochondrial activities. This increases cellular consumption rates and reduces anaerobic glycolysis, breaking down sugar (ATP) to produce energy. Rerouting mitochondrial electron transfer provides neuroprotection against chronic and acute neurological disease. Simplified, this means that methylthioninium chloride increases oxygen to the brain for the cell to breath (mitochondrial respiration) and transfers electrons to the cell to produce energy for function.

Another B-complex vitamin that can be selected for use is vitamin B6 (pyridoxine), which in some examples, can be in the form of pyridoxal 5′ phosphate (PLP). PLP is the active form of vitamin B6 and is a coenzyme for a variety of enzymatic activities, including as a significant coenzyme in transamination reactions, as well as a coenzyme in some decarboxylation, deamination, and racemization reactions of amino acids. Vitamin B6 is used by the body for normal brain development as well as keeping the nervous system and the immune system healthy. Low vitamin B6 levels can also be related to epilepsy. Additionally, deficiencies of vitamin B6 can lead to malabsorption of nutrients in the small intestine, and in some instances, can lead to conditions where healthy red blood cells cannot carry an adequate amount of oxygen to the body (including the brain and nervous system), e.g., sideroblastic anemia. This mechanism and others can cause a subject to experience confusion, depression, brain fog, and a weakened immune system. In some examples, the vitamin B6 can be coupled with one or both of vitamin B9 and/or vitamin B12, which may be in the methylated form.

### Cannabinoids

Cannabinoids are compounds derived from the cannabis plant, with the cannabinoids commonly referred to as CBD (cannabidiol) and/or THC (tetrahydrocannabidiol) receiving much of the attention. However, there are many other cannabinoids with a biogenetic origin that are also present in cannabis plants. One other notable cannabinoid that can provide health benefits is commonly referred to as CBG (cannabigerol), to name one example. It has been found that in some examples of the present disclosure, the inclusion of CBD, and in some cases CBG, can provide benefits to the methylthioninium salt-containing compositions described herein, as neither CBD nor CBG are psychoactive and both have anti-inflammatory properties and are beneficial for central nervous system health, e.g., brain health.

CBD has been shown to be effective for treating depression, panic disorders, mood disorders, psychosis, PTSD-related disorders, etc. CBD in particular produces neurotransmitters known as endocannabinoids that bind to cannabinoid receptors, and can also be neuroprotective against a variety of neurological disorders, with some data indicating efficacy against Parkinson's disease, Huntington's disease, motor disorders, e.g., cerebral palsy, etc. Regarding CBG, this compound can bind to both CB1 and CB2 receptors within the endocannabinoid system (ECS) of the body, which is responsible for maintaining homeostasis of various systems within the body via many receptor sites of the organs, central nervous system, digestive system, immune system, reproductive system, etc. With some similarities and some differences with respect to treatment efficacy of disorders related to the central nervous system, the presence of CBD and/or CBG in some instances can uniquely enhance the therapeutic properties of the methylthioninium salt-containing compositions of the present disclosure. Example central nervous system disorders where a subject may benefit from the presence of one or both of these cannabinoids include autism spectrum disorders (ASD), such as Asperger's syndrome, pervasive developmental disorder (PDD-NOS), autistic disorder, childhood disintegrative disorder, etc., as well as the various attention disorders, such as attention deficit hyperactivity disorder (ADHD), attention deficit disorder (ADD), etc. In accordance with examples herein, if both CBD and CBG are used, they may be included in the methylthioninium salt-containing compositions at from about 1:1 to about 10:1, from about 1.5:1 to about 8:1, from about 2:1 to about 6:1, from about 2:1 to about 4:1, or from about 6.5:3 to about 7.5:3.

### Vitamin E

Vitamin E is an antioxidant that helps protect cells from damage caused by free radicals. Notably, vitamin E may have neuroprotective effects. Vitamin E is one of the fat-soluble vitamins, and as there are only a few fat-soluble vitamins, vitamin E is uniquely positioned to scavenge free radicals that can cause cellular damage, particular at locations such as the cell membrane and other locations where fats may be concentrated. Vitamin E can also enhance immune function and prevent clots from forming in the arteries of the heart. As vitamin E has neuroprotective effects and is a good lipophilic free radical scavenger, it can complement the actions of methylene blue in the context of treating neurodegenerative diseases or conditions involving oxidative stress.

### Manganese

Manganese is an essential mineral that plays a role in various physiological processes, including metabolism, bone formation, and antioxidant defense. Manganese antioxidant properties can be used, for example, to enhance the overall antioxidant capacity when used alongside an appropriate dose of methylthioninium chloride and other compounds that may be present. As the human body does not make manganese, it is often provided by food and/or supplementation, as it is responsible for breaking down carbohydrates, protein, cholesterol, etc., as well as assisting enzymes in building bones. In certain examples, manganese can be provided by a mineral source such as manganese citrate, manganese gluconate, manganese ascorbate, or manganese amino acid chelate. Typically, when calculating the concentration of manganese in a composition, it is the metal content that is calculated for purposes of weight percentages and/or weight ratios with other components that may be present.

### Example Secondary Physiologically Active Compound Combinations

Many of the example methylthioninium salt-containing compositions that can be formulated in accordance with the present disclosure may include multiple secondary physiologically active compounds (in addition to the methylthioninium salt). For example, Table 1 above provides fourteen (14) example methylthioninium salt-containing compositions, nine (9) of which include two or more secondary physiologically active compounds. Table 3 is provided below illustrating those nine (9) combinations as well as several others that can be co-formulated together as a methylthioninium salt-containing compound, or co-administered on a daily basis as separate compositions relative to the methylthioninium salt.

TABLE 3
Example Methylthioninium Salt-containing Compositions with
Multiple Secondary Physiologically Active Compounds

| Comp. ID | Ingredients |
|---|---|
| MB1 | MB^{†}, caffeine, L-theanine |
| MB2 | MB^{†}, green tea, guarana, L-theanine |
| | (opt. with GLP-1 agonist) |
| MB3 | MB^{†}, mineral salts/oxides/chlorides, |
| | hydrogen peroxide (formed in situ) |
| MB4 | MB^{†}, mineral salts/oxides/chlorides, |
| | hydrogen peroxide, colloidal silver |
| MB6 | MB^{†}, glycine, N-acetylcysteine, vitamin B3 (niacinamide) |
| MB7 | MB^{†}, cannabidiol (CBD), vitamin B6, vitamin E, manganese |
| MB12 | MB^{†}, urolithin A, nicotinamide adenine dinucleotide (NAD) |
| MB13 | MB^{†}, urolithin A, pyrroloquinoline quinone |
| MB14 | MB^{†}, urolithin A, pyrroloquinoline quinone, tesofensine |
| MB15 | MB^{†}, caffeine, L-theanine, N-acetylcysteine |
| MB16 | MB^{†}, green tea, guarana, L-theanine, vitamin B3 |
| MB17 | MB^{†}, vitamin C, caffeine, L-theanine |
| MB18 | MB^{†}, green tea, guarana, L-theanine, vitamin B6 |
| MB19 | MB^{†}, mineral salts/oxides/chlorides, |
| | hydrogen peroxide, L-theanine |
| MB20 | MB^{†}, urolithin A, pyrroloquinoline quinone, NAD |
| MB21 | MB^{†}, vitamin C, L-theanine |
| MB22 | MB^{†}, vitamin C, GLP-1 agonist |
| MB23 | MB^{†}, pyrroloquinoline quinone, tesofensine |
| MB24 | MB^{†}, green tea, guarana, L-theanine, black pepper |

MB^{†} refers to methylthioninium chloride in these examples.

Table 4 is also provided below illustrating three (3) liquid nutritional supplement compositions that can be used to enhance the bioavailability of methylthioninium salt and/or other secondary physiologically active compounds, as follows:

TABLE 4
Example Liquid Nutritional Supplement Compositions for
Enhanced Bioavailability of Methylthioninium Salt and/or
Other Secondary Physiologically Active Compounds

| Comp. ID | Ingredients |
|---|---|
| LNS1 | mineral salts/oxides/chlorides, vitamin C, |
| | methylated vitamin B12 |
| LNS2 | mineral salts/oxides/chlorides, vitamin C |
| LNS3 | mineral salts/oxides/chlorides, methylated vitamin B12 |

In addition to those combination therapy examples provided above in Tables 1, 3, and 4, single formulations including compounds from multiple compositions can be formulated or co-administered by combining various examples or portions of various examples. For example, any of these example methylthioninium salt-containing compositions shown in Tables 1, 3, or 4 can be supplemented with other individual compounds from other examples, or multiple full formulations may be appropriate for co-administration. To illustrate, an oral delivery tablet or capsule, such as exemplified at MB1 or MB2, can be co-administered on a daily basis or less than a daily basis with one of the liquid suspension oral formulations, e.g., MB3 or MB4, for example. Daily dosages may be achievable in some instances by alternating various methylthioninium salt-containing compositions.

### Methylthioninium Salt, Caffeine, and L-Theanine

In more specific detail, the methylthioninium salt-containing compositions can include methylthioninium salt, e.g., methylene blue, caffeine, and L-theanine (See MB1-MB2 and MB15-MB18 of Table 3). The methylthioninium salt and the caffeine can be present at a weight ratio from about 1:75 to about 1:4, from about 1:50 to about 1:5, from about 1:35 to about 1:10, or from about 1:25 to about 1:15. The methylthioninium salt and the L-theanine can be present at a weight ratio from about 1:200 to about 1:10, from about 1:100 to about 1:15, from about 1:75 to about 1:20, or from about 1:50 to about 1:25. The caffeine and the L-theanine can be present at a weight ratio from about 1:5 to about 1:1, from about 1:4 to about 1:1, from about 1:3 to about 1:1.5, or about 2:1.

The combination of methylthioninium salt, caffeine, and L-theanine provides some unique properties when co-formulated together, which can enhance or expand the benefits of the individual ingredients if dosed alone. For example, while caffeine is a stimulant, L-theanine promotes relaxation and reduces stress levels. In the body, with both present at appropriate concentrations, these two compounds can balance out the effects of one another, increasing the productivity and improving mental clarity of a subject taking this combination. Thus, the subject can experience the effects of the stimulant without all of the negative side effects that are sometimes associated with caffeine consumption, e.g., jitters, etc. Additionally, stacking caffeine and L-theanine can work synergistically to increase performance, reduce anxiety, etc., even when performing stressful tasks. In some examples, the caffeine to L-theanine ratio can be about 2:1 for more optimal performance, but ratios outside of this range can likewise be used, as described above. Furthermore, the presence of the methylene blue at a relatively low dose provides the additional benefit of increasing oxygen to the body, and in particular the central nervous system, e.g., brain, as it is capable of passing the blood brain barrier. Furthermore, the methylene blue can assist with preventing dopamine and/or serotonin from breaking down. Thus, increased levels of neurotransmitters can be present, enhancing cognitive performance, memory, focus, learning, mood, and overall well-being.

As an oral dosage form, this composition can be prepared in the form of tablets, capsules, soft-gels, powders, liquid suspensions or solutions, or transmucosal oral formulations. An example of a typical daily dose of the methylthioninium salt-containing composition can include from about 1 mg to about 50 mg, from about 1 mg to about 25 mg, or from about 1 mg to about 12 mg of methylthioninium salt, from about 20 mg to about 450 mg or from about 40 mg to about 225 mg of caffeine, and from about 25 mg to about 900 mg or from about 50 mg to about 450 mg of L-theanine. In a more specific example, a typical dose of the methylthioninium salt-containing composition can include from about 1.5 mg to about 5 mg of methylthioninium salt, from about 25 mg to about 125 mg of caffeine, and from about 50 mg to about 250 mg of L-theanine. In a more specific example, the methylthioninium salt-containing composition can include from about 2 mg to about 4 mg of methylthioninium salt, from about 25 mg to about 75 mg of caffeine, and from about 50 mg to about 150 mg of L-theanine. Higher daily intakes of some or all of these compounds can likewise be taken in divided doses throughout the day in some examples. For example, depending on the treatment, oral delivery can be used to treat a subject via from about 1 to about 4 dosages per day.

The caffeine and/or the L-theanine can be provided as pure substances, such as extracts in order to generate more precise dosing, but in some examples, the caffeine and/or the L-theanine can be provided by certain natural components that contain caffeine and/or L-theanine (as well as other compounds that may also be present in the natural components). For example, in some examples, at least a portion of the caffeine and L-theanine can be provided by green tea and/or at least a portion of the caffeine can be provided by guarana. Example methylthioninium salt-containing compositions containing green tea and/or guarana may contain a weight ratio of methylthioninium salt to green tea from about 1:3 to about 1:25, from about 1:4 to about 1:20, from about 1:5 to about 1:25, from about 1:5 to about 1:15, or about 1:10. These compositions may likewise include a methylthioninium salt to guarana weight ratio from about 1:10 to about 1:40, from about 1:15 to about 1:35, or from about 1:20 to about 1:30, for example. The weight ratio of methylthioninium salt to L-theanine can be from about 1:200 to about 1:10, from about 1:100 to about 1:15, from about 1:75 to about 1:20, or from about 1:50 to about 1:25. Notably, if green tea is present, at least some of the L-theanine may be provided by the presence of the green tea, for example. Furthermore, in some examples, if both green tea and guarana are present, they can be present at a weight ratio from about 1:5 to about 1:1, from about 1:4 to about 1:1.5, or from about 1:3 to about 1:2.

As an oral dosage form, this methylthioninium salt-containing composition that includes green tea and/or guarana to provide at least a portion of its caffeine component can be prepared in the form of tablets, capsules, soft-gels, powders, liquid suspensions or solutions, or transmucosal oral formulations. An example of a typical dose of the methylthioninium salt-containing composition can include from about 1 mg to about 12 mg of methylthioninium salt, from about 10 mg to about 120 mg green tea, from about 20 mg to about 220 mg guarana, and from about 50 mg to about 450 mg of L-theanine. In a more specific example, a typical dose of the methylthioninium salt-containing composition can include from about 1.5 mg to about 5 mg of methylthioninium salt, from about 15 mg to about 75 mg of green tea, from about 40 mg to about 100 mg guarana, and from about 50 mg to about 250 mg of L-theanine. In a more specific example, the methylthioninium salt-containing composition can include from about 2 mg to about 4 mg of methylthioninium salt, from about 20 mg to about 50 mg of green tea, from about 50 mg to about 75 mg of guarana, and from about 50 mg to about 150 mg of L-theanine. Depending on the treatment, oral delivery can be used to treat a subject via from about 1 to about 4 dosages per day, for example.

These methylthioninium salt-containing compositions can be used for any of the various conditions described herein, but can be particularly useful when orally administered to a subject to treat attention disorders, such as attention deficit hyperactivity disorder (ADHD) or attention deficit disorder (ADD), for example. This particular composition can also be used in supporting weight loss as well as for metabolism glucagon-like peptide-1 (GLP-1) enhancement, and can also improve insulin sensitivity and enhance mitochondria health. In further detail, these combinations of methylthioninium salt-containing compositions can be used for metabolic ignition, promoting calorie burning and increased energy expenditure, which can be particularly the case in compositions containing the guarana. In fact, the synergy between mitochondrial support provided by methylthioninium salt and the natural caffeine and other components of guarana can boost mitochondrial health, aid in efficient nutrient utilization, and foster overall cell vitality. On the other hand, green tea provides a good source of catechins, such as EGCG, and works synergistically with guarana to even further augment weight loss, stimulate fat oxidation, regulate appetite, improve insulin sensitivity, etc. The thermogenic properties of green tea combined with the metabolic boost provided by guarana can promote efficient fat utilization, calorie burning, and a more streamlined metabolism, e.g., thermogenesis and fat utilization. In further detail, both guarana and green tea work synergistically together to combat oxidative stress due to their individual unique antioxidant properties, providing an oxidative stress defense that is better than either compound when used alone. In other examples, pairing caffeine (such as from guarana) with L-theanine, can work synergistically together to provide an energy boost accompanied by mental clarity and focus. The L-theanine can also reduce the jittery effect of caffeine that some subjects experience.

In further detail, the methylthioninium salt-containing compositions can include additional compounds, including any of the other compounds described herein, and in some more specific examples, may further include black pepper, vitamin B6, vitamin E, manganese, a cannabinoid, nicotine, or a combination thereof.

In accordance with this, a method of treating a subject can include orally administering the methylthioninium salt-containing composition described herein which includes methylthioninium salt, caffeine, and L-theanine. The caffeine may, in some examples, be provided in part or in whole by green tea and/or guarana. These methylthioninium salt-containing compositions can be used for any of the various conditions described herein, but can be particularly useful when orally administered to a subject to treat attention disorders, such as attention deficit hyperactivity disorder (ADHD) or attention deficit disorder (ADD), for example. They can also be used to ameliorate brain fog or dementia, and/or can be used to treat Lyme disease or chronic fatigue, for example.

With more specific respect to long COVID or vaccine-induced spike protein damage, and/or dementia, the one or more of the methylthioninium salt-containing compositions described herein can be administered to reduce symptoms and/or heal the body and/or brain. As an example, a composition that includes methylthioninium salt, caffeine, and L-theanine can be taken first thing in the morning to jump start the brain. Other methylthioninium salt-containing compositions described herein can likewise be administered, such as the composition that includes methylthioninium salt and vitamin C (described hereinafter), or the composition that includes methylthioninium salt, glycine, N-acetylcysteine, and niacinamide (also described hereinafter), among others. In some examples, when treating long COVID or vaccine-induced spike protein damage in particular, the subject may benefit initially from the boost of energy in the morning from one or more of these methylthioninium salt-containing compositions in combination with a composition that includes methylthioninium salt, mineral oxides/salts, and hydrogen peroxide (described hereinafter), which may further be formulated to also include colloidal silver in some examples.

### Methylthioninium Salt, Mineral Salts/Oxides/Chlorides, and Hydrogen Peroxide

The methylthioninium salt-containing compositions can be in the form of a liquid suspension (for oral delivery), and can include water, methylthioninium chloride, mineral blend of salts, oxides, and/or chlorides, and hydrogen peroxide (See MB3-MB4 and MB19 of Table 3). The methylthioninium chloride and the mineral blend of salts, oxides, and/or chlorides can be present at a weight ratio from about 100:1 to about 1:4, from about 40:1 to about 2:1, from about 30:1 to about 3:1, or from about 25:1 to about 5:1.

As an example, a single dose of this particular methylthioninium salt-containing composition, e.g., dosed at about 0.25 mL to about 5 mL or about 0.5 mL to about 2.5 mL, can include from about 1 mg to about 50 mg or about 1 mg to about 20 mg of the methylthioninium chloride per mL, and from about 0.2 mg to about 20 mg or from about 0.2 mg to about 10 mg of the mineral blend of salts, oxides, and/or chlorides per milliliter (mL). Higher daily intakes of some or all of these compounds can likewise be taken in divided doses throughout the day in some examples. The mineral blend of salts, oxides, and/or chlorides in some examples may include magnesium chloride, potassium chlorate, sodium chloride, and/or magnesium oxide. In some examples, the methylthioninium chloride to the mineral blend of salts, oxides, and/or chlorides (in combination) may be present at from about 0.01 wt % to about 0.5 wt %, from about 0.025 wt % to about 0.05 wt %, or from about 0.1 wt % to about 0.3 wt %. The magnesium chloride and/or the sodium chloride, for example, can provide the benefits of a digestive aid, as the chloride is used in the stomach in the formation of stomach acid, e.g., HCl. The potassium chlorate can contribute as a strong oxidizer and can benefit oral health care, for example. The magnesium oxide can provide the benefits of an antacid compound.

In further detail, these methylthioninium salt-containing compositions can include additional compounds, including any of the other compounds described herein, and in some more specific examples, may further include colloidal silver, e.g., from about 5 ppm to about 50 ppm or from about 10 ppm to about 40 ppm with a particle size from about 3 nm to about 20 nm or from about 5 nm to about 15 nm. Other additional compounds that can be present include black pepper, vitamin B6, vitamin E, manganese, a cannabinoid, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

In accordance with this, a method of treating a subject can include orally administering the methylthioninium salt-containing composition described herein which includes water, methylthioninium chloride, mineral blend of salts, oxides, and/or chlorides, and hydrogen peroxide. These methylthioninium salt-containing compositions can be used for any of the various conditions described herein, but in particular can be particularly useful when orally administered to a subject, e.g., typically daily, to treat long COVID or vaccine-induced spike protein damage, ameliorate brain fog, treat Lyme disease, and/or some of the other conditions outlined herein.

Methylthioninium Salt, Vitamin C, and One or More of Caffeine, L-Theanine, and/or GLP-1 Agonist

In some examples, the methylthioninium salt-containing compositions can include methylthioninium chloride and vitamin C (ascorbic acid and/or ascorbate) (See MB5 in Table 1). However, other secondary physiologically active compounds can be included as well, such as by way of example, caffeine and/or L-theanine (See MB17 or MB21 of Table 3). The caffeine, if present, may be sourced from green tea and/or guarana, for example. Other compounds may also be included, such as vitamin B6, vitamin E, manganese, a cannabinoid, nicotine, and/or other compounds that may assist with the efficacy of the methylthioninium salt-containing compositions of the present disclosure. Ratios may vary, but in some examples, the weight ratio of the methylthioninium salt to vitamin C can be from about 1:2 to about 1:10, 1:2.5 to about 1:7.5, or from about 1:4 to about 1:6.

As an oral dosage form, this composition can be prepared in the form of tablets, capsules, soft-gels, powders, liquid suspensions or solutions, or transmucosal oral formulations. An example of a typical single or divided daily dose of the methylthioninium salt-containing composition can include from about 1 mg to about 50 mg, from about 2.5 mg to about 25 mg, from about 7.5 mg to about 25 mg or from about 10 mg to about 20 mg of methylthioninium chloride. The single or divided daily dose of the vitamin C can be from about 50 mg to about 1000 mg, from about 50 mg to about 500 mg, from about 50 mg to about 250 mg, from about 50 mg to about 150 mg, from about 50 mg to about 100 mg, or from about 60 mg to about 90 mg (including ascorbic acid and/or ascorbate). Higher daily intakes of some or all of these compounds can likewise be taken in divided doses throughout the day in some examples. The L-theanine, on the other hand, can be dosed at from about 50 mg to about 450 mg of L-theanine. If green tea and/or guarana are included, they may be dosed at from about 10 mg to about 120 mg green tea and/or from about 20 mg to about 220 mg guarana, for example. In a more specific example, a typical dose of the methylthioninium salt-containing composition can include from about 1.5 mg to about 5 mg of methylthioninium salt, from about 15 mg to about 75 mg of green tea, from about 40 mg to about 100 mg guarana, and from about 50 mg to about 250 mg of L-theanine. In a more specific example, the methylthioninium salt-containing composition can include from about 2 mg to about 4 mg of methylthioninium salt, from about 20 mg to about 50 mg of green tea, from about 50 mg to about 75 mg of guarana, and from about 50 mg to about 150 mg of L-theanine. Depending on the treatment, oral delivery can be used to treat a subject via from about 1 to about 4 dosages per day, for example. These methylthioninium salt-containing compositions (or co-administration of these compounds) can be particularly effective for treating early-stage cognitive decline, e.g., dementia, Alzheimer's disease, etc., and may also provide cognitive benefits and even weight loss support.

In some more specific examples, MB5 of Table 1 or MB 17 of Table 3 may be co-administered with a GLP-1 medication (or agonist), such as dulaglutide, exenatide, liraglutide, lixisenatide, semaglutide, tirzepatide, or the like. MB22 of Table 3 illustrates an example combination that includes the GLP-1 agonist. Generally, GLP-1 agonists can be used to treat diabetes or subjects that may be pre-diabetic and/or overweight by stimulating the release of insulin and/or lower blood sugar levels. By combining a GLP-1 agonist with this or other methylthioninium salt-containing compositions described herein, additional weight loss along with enhanced energy may be experienced, for example.

### Methylthioninium Chloride, Glycine, N-Acetylcysteine, and Vitamin B3

In some examples, the methylthioninium salt-containing compositions can include methylthioninium chloride, glycine, N-acetylcysteine, and a vitamin B3 (See MB6 in Table 3). In this example, the methylthioninium chloride and the glycine can be present at a weight ratio from about 1:3 to about 1:25 or from about 1:5 to about 1:15, the methylthioninium chloride and the N-acetylcysteine can be present at a weight ratio from about 1:3 to about 1:25 or from about 1:5 to about 1:15, and/or the methylthioninium chloride and the vitamin B3 can be present at a weight ratio from about 1:2 to about 1:12 or from about 1:2 to about 2:1. In a more detailed example, the weight ratio of glycine to N-acetylcysteine can be from about 1:3 to about 3:1, from about 1:2 to about 2:1, or about 1:1. The vitamin B3, for example, can be in the form of nicotinic acid (flushing-type niacin) and/or niacinamide. In some examples, the vitamin B3 consists or consists essentially of niacinamide.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 1 mg to about 50 mg, from about 2 mg to about 15 mg, or from about 2.5 mg to about 10 mg of methylthioninium chloride. These dosages can be delivered as a single dose or via multiple divided doses (multiple “single” doses can be administered throughout the day), or this formulation can be stacked with other methylthioninium salt-containing compounds described herein. In further detail, the methylthioninium salt-containing composition can include a single dose or multiple divided daily dosages totaling from about 20 mg to about 100 mg or from about 20 mg to about 75 mg glycine, from about 25 mg to about 100 mg or from about 25 mg to about 75 mg N-acetylcysteine, and/or from about 5 mg to about 50 mg or from about 15 mg to about 40 mg of vitamin B3, e.g., niacinamide. Higher daily intakes of some or all of these compounds can likewise be taken in divided doses throughout the day in some examples.

In further detail, these methylthioninium salt-containing compositions can include additional compounds, including any of the other compounds described herein, and in some more specific examples, may further include black pepper, vitamin B6, vitamin E, manganese, a cannabinoid, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

A method of treating a subject can include orally administering the methylthioninium salt-containing composition described herein which includes methylthioninium chloride, glycine, N-acetylcysteine, and vitamin B3. These methylthioninium salt-containing compositions can be used for any of the various conditions described herein, but can be particularly useful when orally administered to a subject, e.g., typically daily, to treat cognitive conditions, such as those related to memory, behavior, thinking, etc. Examples of conditions for which this more specific composition can be beneficial include the treatment/amelioration of general cognitive decline (pre-emptively or after onset) and/or the treatment of traumatic brain injury. In further detail, as the methylthioninium chloride is a strong antioxidant, it can be dosed to reduce oxidative stress, enhance energy levels, and improve cognition. Methylthioninium chloride can also protect the brain from damage caused by beta-amyloid, a protein that may contribute to the development of Alzheimer's disease. In further detail, glycine, which is the simplest amino acid, can also have neuroprotective properties and can assist with improving cognition, also assisting with the prevention of the formation of beta-amyloid plaques. Furthermore, vitamin B3, and particularly niacinamide in some examples, can be protective of the central nervous system, e.g., brain and nervous system, including protection against beta-amyloid accumulation and other harmful brain changes associated with dementia. Thus, methylthioninium chloride, the glycine, and the vitamin B3 can work together at least in the protection against beta-amyloid accumulation, for example. Furthermore, N-acetylcysteine is also a powerful antioxidant effective at protecting the central nervous system, with supplementation providing improved cognition, memory, etc., particularly with subjects suffering from one form or another of dementia.

### Methylthioninium Chloride, Cannabinoids, Vitamin B6, Vitamin E, and Manganese

In some examples, the methylthioninium salt-containing compositions can include methylthioninium chloride, a cannabinoid, vitamin B6, vitamin E, and manganese (See MB7 in Table 3). In some examples, the methylthioninium chloride and the cannabinoid can be present at a weight ratio from about 1:3 to about 1:10 or from about 1:4 to about 1:7, the methylthioninium chloride and the vitamin B6 can be present at a weight ratio from about 10:1 to about 2:1 or from about 7:1 to about 3:1, the methylthioninium chloride and the vitamin E can be present at a weight ratio from about 1:3 to about 3:1 or from about 1:2 to about 1:1, and the methylthioninium chloride and the manganese metal content can be present at a weight ratio from about 10:1 to about 2:1 or from about 7:1 to about 2.5:1.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 1 mg to about 50 mg, from about 1 mg to about 25 mg, from about 1 mg to about 12 mg, or from about 2 mg to about 10 mg of methylthioninium chloride. The cannabinoid(s) can be included at from about 10 mg to about 100 mg, from about 10 mg to about 50 mg, or from about 15 mg to about 40 mg. The vitamin B6 can be dosed at from about 0.2 mg to about 6 mg, from about 0.2 mg to about 3 mg, or from about 0.5 mg to about 2 mg. The vitamin E can be included for daily or divided daily doses totaling from about 3 mg to about 30 mg, from about 3 mg to about 15 mg, or from about 5 mg to about 10 mg. The manganese (by metal content) can be included at from about 0.5 mg to about 8 mg, from about 0.5 to about 4 mg, or from about 1 mg to about 3 mg. Again, higher daily intakes of some or all of these compounds can likewise be taken in divided doses throughout the day in some examples.

These compositions can be effective for treating/preventing epilepsy and/or seizures, and can also be effective for promoting or treating sleep disorders, for example. The methylthioninium chloride, for example, can act as a redox agent and mitochondrial enhancer. By improving mitochondrial function and energy production in neurons, it enhances their resilience to excitotoxicity and oxidative stress, both implicated in epilepsy. Methylthioninium chloride also has ion channel-blocking properties, potentially stabilizing neuronal membranes and reducing hyperexcitability. The cannabinoid(s), e.g., CBD, can interact with the endocannabinoid system, modulating neurotransmitter release and ion channel activity, which can reduce neuronal hyperexcitability associated with seizures. Furthermore, the cannabinoid CBD can be anti-inflammatory and neuroprotective, which can assist with mitigating seizure activity. Vitamin B6, particularly in the form of pyridoxal phosphate (PLP), is a cofactor for enzymes involved in neurotransmitter synthesis and metabolism, such as glutamate decarboxylase, which converts glutamate to GABA, an inhibitory neurotransmitter. Adequate levels of vitamin B6 can support GABAergic transmission, helping to dampen excessive neuronal firing and reduce seizure susceptibility. Furthermore, vitamin E is a potent antioxidant that scavenges free radicals and protects cell membranes from oxidative damage. Oxidative stress is implicated in epileptogenesis, and the presence of vitamin E in the compositions described herein can assist with mitigating the effects and reducing the frequency of seizures. Furthermore, manganese is a cofactor for various enzymes, including glutamine synthetase, which converts glutamate to glutamine. Glutamine assists with maintaining the glutamate-glutamine cycle and regulating glutamate levels in the brain. Dysregulation of glutamate homeostasis is associated with neuronal hyperexcitability and seizures. Adequate manganese levels support this cycle, potentially reducing glutamate-mediated excitotoxicity and seizure occurrence. By combining these components in a single formulation or composition for orally dosing subjects can promote a synergistic effect in epilepsy management, with the cannabinoid CBD and methylthioninium chloride targeting hyperexcitability through alternative pathways (both compounds having a different mechanism of action and thus a stronger effect). Furthermore, the presence of vitamins B6 and E, along with manganese, can support neurotransmitter balance (vitamin B6), and can mitigate oxidative stress (vitamin E, and regulate glutamate levels (manganese), thus providing a more effective approach for managing epilepsy and seizures than any of these compounds alone in most instances.

In further detail, these methylthioninium salt-containing compositions can include additional compounds, including any of the other compounds described herein, and in some more specific examples, may further include black pepper, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

In accordance with this, a method of treating a subject can include orally administering the methylthioninium salt-containing composition described herein which includes methylthioninium chloride, a cannabinoid, vitamin B6, vitamin E, and manganese. These methylthioninium salt-containing compositions can be used for any of the various conditions described herein, but can be particularly useful when orally administered to a subject, e.g., typically daily, to treat/prevent seizures, epilepsy, and sleep disorders, e.g., provide sleep support, as described above.

### Physiological Therapies

As briefly mentioned, there are multiple physiological therapies that can assist with providing a therapeutic effect in a subject that is being co-administered a methylthioninium salt and one or more of the secondary physiologically active compounds as described herein. Example physiological therapies may include those therapies listed previously in Table 2, among others. The various categories of therapies include electromagnetic therapies, thermal therapies, oxygenation therapies, to name a few.

### A. Electromagnetic Therapies

In accordance with the present disclosure, various electromagnetic therapies can be utilized with efficacy, including therapies such as red-light therapy, pulse electromagnetic field therapy, or the like. Red-light therapy can, for example, improve cognitive function, including executive function, memory, and visual attention. It can also reduce oxidative stress, restore memory function, and/or can also reduce beta-amyloid plaques in the brain. Combining red-light light therapy with the co-administration of methylthioninium salts and one or more of the secondary physiologically active compounds described herein can offer an additive or even a synergistic benefit for mitochondrial function and overall health.

Red-light Therapies with Co-administration of Methylthioninium Salt and Secondary Physiological Compound(s)

Red-light therapy can be conducted for a period of time of about 5 minutes to about 60 minutes, from about 5 minutes to about 45 minutes, from about 5 minutes to about 30 minutes, from about 10 minutes to about 60 minutes, from about 10 minutes to about 45 minutes, from about 10 minutes to about 30 minutes, from about 15 minutes to about 60 minutes, or from about 15 minutes to about 45 minutes. The red-light therapy can deliver red-light from the visible spectrum to the infrared spectrum, e.g., from about 600 nm to about 1400 nm. Example visible spectrum red-light therapy can delivery visibly red-light at from about 600 nm to about 750 nm. Example near infrared or infrared spectrum red-light therapy can deliver the “red-light” at from greater than about 750 nm to about 1400 nm. In some examples, it may be beneficial to select one or a few peak energy wavelengths that may be particularly suited for treating conditions or improving specific health or overall health goals. For example, combining 630 nm and 660 nm or combining a red-light wavelength, e.g., 660 nm, with a near infrared wavelength, e.g., 830 nm and/or 850 nm, can be effective red-light therapy treatment. The intensity of the red-light used for the red-light therapy may vary, based on wavelength, body coverage, etc. Regarding the power level of the red-light therapy, example ranges can be from about 250 milliwatts (mW) to about 80 watts (W), from about 500 mW to about 80 W, from about 1 W to about 80 W, from about 5 W to about 80 W, from about 250 mW to about 20 W, from about 500 mW to about 20 W, or from about 1 W to about 20 W, for example. Intensities of red-light therapy (joules per second per square meter) depend on the period of time of treatment.

In some examples, mitochondrial enhancement or health improvement can be achieved using red-light therapy combined with methylthioninium salts and secondary physiologically active compounds. To illustrate, red-light therapy can stimulate mitochondrial function by increasing ATP production and cellular energy. For a specific example, red-light therapy set at a wavelength from about 630 nm to about 675 nm and/or from about 800 nm to about 1080 nm, e.g., about 660 nm, about 670 nm, about 830 nm, and/or about 850 nm, can stimulate energy production of the mitochondria, leading to increased consumption of glucose. In some instances, the increased consumption of glucose can have the added benefit of reducing blood glucose levels and ameliorating glucose spiking. Methylene blue can provide an alternative electron donor in the mitochondrial electron transport chain, providing an improved pathway for increasing ATP production and enhancing mitochondrial function. Regarding the secondary physiologically active compound, urolithin A can be selected for enhancement of mitophagy (removal of damaged mitochondria) and for the improvement of mitochondrial function. Pyrroloquinoline quinone (PQQ) can be selected for enhancing mitochondrial biogenesis and function. Green tea (polyphenols) can be selected for stimulating mitochondrial biogenesis as well. Other secondary physiologically active compounds can also be selected as well.

Red-light therapy combined with the co-administration of methylthioninium salt and one or more secondary physiologically active compounds can also provide antioxidant and anti-inflammatory effects, and in some instances, can support wound/injury healing and pain relief. For example, red-light therapy can reduce inflammation and oxidative stress. Example wavelengths for inflammation can be from about 630 nm to about 675 nm and/or from about 800 nm to about 1080 nm, e.g., about 630 nm, about 660 nm, about 670 nm, about 760 nm, about 810-830 nm, about 900 nm, and/or about 980 nm. In addition to the anti-inflammatory and antioxidant properties of methylene blue, the secondary physiologically active compound that can be used includes vitamin C, green tea (polyphenols), and/or guarana, for example, all of which have antioxidant properties.

In further detail, red-light therapy combined with the co-administration of methylthioninium salt and one or more secondary physiologically active compounds can also provide neuroprotection. Red-light therapy can enhance cognitive function and also provide neuroprotection and brain health, even when treating a subject with a neurodegenerative disease. Example wavelengths for inflammation can be from about 630 nm to about 675 nm and from about 800 nm to about 1080 nm, e.g., about 627 nm, about 630 nm, about 660 nm, about 670 nm, about 800 nm, about 810 nm, about 820 nm, and/or about 1070 nm. Near infrared wavelengths can penetrate the skull better than longer wavelengths, so in some instances, it can be beneficial to use a near infrared wavelength in the treatment when targeting brain tissue. Methylene blue also provides neuroprotective properties, particularly when combined with pyrroloquinoline quinone (PQQ) as it can reduce inflammation and improve mitochondrial function in the brain, for example.

Red-light therapy combined with the co-administration of methylthioninium salt and one or more secondary physiologically active compounds can also improve skin and hair health. For example, the red-light therapy can improve complexion, reduce wrinkles, and promote collagen production. Example wavelengths for inflammation can be from about 630 nm to about 675 nm and from about 800 nm to about 1080 nm, e.g., about 630 nm, about 640 nm, about 650 nm, about 660 nm, about 670 nm, about 830 nm, about 855 nm, and/or about 860 nm. In addition to the delivery of methylene blue in connection with the red-light therapy, green tea can be used to accelerate skin rejuvenation, for example.

Red-light therapy combined with the co-administration of methylthioninium salt and one or more secondary physiologically active compounds can also enhance energy and performance. For example, the red-light therapy can enhance mitochondrial function resulting in enhanced energy. Example wavelengths for inflammation can be from about 630 nm to about 675 nm and from about 800 nm to about 1080 nm, e.g., about 630 nm, about 640 nm, about 660 nm, about 670 nm, about 750 nm, about 850 nm, about 900 nm, and/or about 980 nm. In addition to the delivery of methylene blue in connection with the red-light therapy, guarana can be co-administered therewith to enhance energy levels.

In other examples, red-light therapy combined with the co-administration of methylthioninium salt and one or more secondary physiologically active compounds can also provide anti-aging effect. For example, red-light therapy can stimulate stem cells and promote tissue repair. Example wavelengths for inflammation can be from about 600 nm to about 675 nm and from about 800 nm to about 1080 nm, e.g., about 605 nm, about 630 nm, about 640 nm, about 650 nm, about 660 nm, about 670 nm, about 830 nm, about 855 nm, and/or about 860 nm. Methylene blue, for example, can assist with combatting oxidative stress associated with aging. In addition to the delivery of methylene blue in connection with the red-light therapy, urolithin A is an example secondary physiologically active compound that can be used to assist with ameliorating oxidative stress associated with aging.

Pulsed Electromagnetic Field (PEMF) Therapies with Co-administration of Methylthioninium Salt and Secondary Physiological Compound(s)

Pulsed electromagnetic field (PEMF) therapy can be used for many purposes, such as managing inflammation, e.g., inflammation associated with Alzheimer's disease, activating brain regions involved in memory circuits, slowing cognitive decline, etc. PEMF can also enhance mitochondrial function by enhancing nutrient flow into the cells and enhancing mitochondrial efficiency, which can lead to tissue and overall bodily function health. PEMF therapy, using pulse repetition frequency resonances, can promote the opening of the cell membrane channels to allow nutrients in and waste out, which can lead to restoration of cellular balance. Combining PEMF therapy with the co-administration of methylthioninium salt and a secondary physiological compound(s) can provide additive or even synergistic benefits, including for mitochondrial function and/or overall health. PEMF therapy can be conducted for a period of time from about 5 minutes to about 60 minutes, from about 5 minutes to about 45 minutes, from about 5 minutes to about 30 minutes, from about 10 minutes to about 60 minutes, from about 10 minutes to about 45 minutes, from about 10 minutes to about 30 minutes, from about 15 minutes to about 60 minutes, or from about 15 minutes to about 45 minutes. PEMF therapy, for example, can be delivered at a variety of flux densities or magnetic field intensities (or “intensity”), e.g., from about 10 milligauss to about 5000 gauss (or more). High intensity PEMF can range in intensity from above about 1000 gauss to about 5,000 gauss. Low intensity PEMF can also be effective for treatment, which can range from as low as about 10 milligauss up to about 1000 gauss, but more typically from about 200 milligauss to about 2 gauss or from about 200 milligauss to about 1 gauss. Intensities between these high intensity and low intensity ranges can also be used, e.g., from about 10 gauss to about 1000 gauss.

When referring to an electromagnetic “frequency” in the context of PEMF, there are two contexts that can be used, namely the “field frequency” or the “pulse repetition frequency.” With field frequency, there are generally multiple categories, including Ultra Low Frequency (ULF) at less than about 3 Hz, Extremely Low Frequency (ELF) at from about 3 Hz to about 30 kHz), and Very Low Frequency (VLF) at from about 30 KHz to about 300 KHz. These are not typically the “frequencies” referred when referring to PEMF. As PEMF refers to pulsed electromagnetic field, the frequency referred to typically relates more directly to the frequency of the pulse of the electromagnetic energy. Thus, the electromagnetic energy has a field frequency (the frequency of the electromagnetic signal itself) and a pulsed repetition frequency (the frequency at which the electromagnetic energy is pulsed on and off). The pulsed repetition frequencies as used herein can typically be from greater than 0 Hz to about 50 Hz, but more often from about 1 Hz to about 30 Hz, depending on the target of the therapy. In accordance with this, electromagnetic signal or energy used for PEMF typically includes a continuous train of sine waves and/or other signal shapes (the field frequency) that are intermittently turned on and off (the pulse repetition frequency). Notably, the field frequency itself may be relatively high, often in the kHz range (or even MHz range). For example, a high field frequency signal can be delivered via PEMF therapy at a Schumann resonance pulse repetition frequency, such as at from about 7.6 Hz to about 8 Hz, or about 7.8 Hz. Various other pulse repetition frequencies can be selected within those ranges for treatment of various conditions, with some pulse repetition frequencies being better suited for some conditions, and other pulse repetition frequencies being better suited for other conditions. Example frequencies are included herein in the context of various treatments, but it is noted that those ranges can be applicable to other treatments not specifically indicated, as they each fall within the broad range of from of greater than 0 Hz to about 50 Hz.

Mitochondrial enhancement can be achieved using PEMF therapy combined with methylthioninium salts and a secondary physiologically active compound(s). To illustrate, PEMF therapy can stimulate mitochondrial function by increasing ATP production and cellular energy. For a specific example, PEMF therapy set at frequencies such as from about 1 Hz to about 30 Hz, from about 1 Hz to about 8 Hz or from about 13 to about 30 Hz can be delivered at low intensities, e.g., from about milligauss to about 1000 gauss or from about 200 milligauss to about 2 gauss. Methylene blue can provide an alternative electron donor in the mitochondrial electron transport chain, providing an improved pathway for increasing ATP production and enhancing mitochondrial function. Regarding the secondary physiologically active compound, green tea (polyphenols) can stimulate biogenesis, urolithin A can enhance mitophagy (removal of damaged mitochondria) and improve mitochondrial function, and pyrroloquinoline quinone (PQQ) can enhance mitochondrial biogenesis and function. Other secondary compounds that can be used for enhancing mitochondrial function include N-acetyl cysteine (NAC), glycine, vitamin B3, e.g., niacinamide, among others, as they can support the reduction of oxidative stress and enhance mitochondrial efficiency. For example, N-acetylcysteine is a precursor to glutathione (a strong antioxidant), and thus can assist with replenishing glutathione levels, reduce oxidative stress, and generally protect cells from damage. Glycine is the simplest amino acid, but plays a large role in many metabolic processes, such as collagen production which is involved in tissue repair and skin health. Glycine also supports mitochondrial function and reduces oxidative stress. Niacinamide (and to a lesser extent nicotinic acid) can enhance cellular energy production and is involved in DNA repair. Niacinamide is a precursor to nicotinamide adenine dinucleotide (NAD), which is a coenzyme involved in cellular energy production. Vitamin B3 (niacinamide or nicotinic acid) also plays a role in DNA repair, which contributes to cellular health and the prevention of mutations.

Cellular repair, regeneration, and health can be achieved using PEMF therapy combined with methylthioninium salts and a secondary physiologically active compound(s). For example, PEMF therapy supports cellular repair and regeneration by elevating ATP production. To illustrate, PEMF therapy can stimulate mitochondrial function by increasing ATP production and cellular energy. For a specific example, PEMF therapy set at frequencies such as from about 1 Hz to about 30 Hz can be delivered at low intensities, e.g., from about 10 milligauss to about 1000 gauss or from about 200 milligauss to about 2 gauss. Methylene blue can provide an alternative electron donor in the mitochondrial electron transport chain, providing an improved pathway for increasing ATP production and enhancing mitochondrial function. Regarding the secondary physiologically active compound, vitamin C and/or green tea (catechins), which have antioxidant properties, can enhance the cellular repair and regeneration by protecting cells from oxidative damage.

Reducing inflammation, or an anti-inflammatory effect, can be achieved using PEMF therapy combined with methylthioninium salts and a secondary physiologically active compound(s). For example, PEMF therapy supports cellular repair and regeneration by elevating ATP production. To illustrate, PEMF therapy can stimulate mitochondrial function by increasing ATP production and cellular energy. For a specific example, PEMF therapy set at frequencies such as from about 1 Hz to about 30 Hz can be delivered at low intensities, e.g., from about 10 milligauss to about 1000 gauss or from about 200 milligauss to about 2 gauss. Methylene blue can provide an alternative electron donor in the mitochondrial electron transport chain, providing an improved pathway for increasing ATP production and enhancing mitochondrial function. Regarding the secondary physiologically active compound, green tea (polyphenols) and guarana can enhance the anti-inflammatory properties provide by the co-administration of methylene blue along with the PEMF therapy.

Other benefits of PEMF therapy combined with methylthioninium salts and a secondary physiologically active compound(s) may include enhanced neuroprotection and cognition, e.g., from about 1 Hz to about 30 Hz, enhanced energy and performance, e.g., from about 1 Hz to about 30 Hz, improved sleep, e.g., from about 0.5 Hz to about 7 Hz or from about 1 Hz to about 5 Hz, stress reduction, e.g., from about 0.5 Hz to about 7 Hz or from about 1 Hz to about 5 Hz, pain management, e.g., from about 5 Hz to about 20 Hz or from about 5 Hz to about 13 Hz, improved blood circulation, e.g., from about 2 Hz to about 6 Hz or about 20 Hz, among others. Similar secondary physiologically active compounds for these conditions or treatments can be used as described previously with respect to red-light therapy. With specific reference to stress reduction and or improved sleep (or sleep disorder treatment), the adaptogenic properties of some compounds, such as guarana, green tea, and others can enhance the efficacy of the PEMF therapy.

### B. Thermal Therapies

Various thermal therapies, particularly full body thermal therapies, can include cold exposure therapy, e.g., cold plunge, cold water immersion, cryotherapy, etc., and/or heat exposure therapy, e.g., sauna therapy. Some of the benefits of cold exposure therapy, for example, include the easing of sore muscles, enhancing focus, enhancing sleep, decreasing inflammation, etc. Sauna therapy can likewise be used for achieving similar general benefits. However, there are other physiological benefits to cold exposure therapy and/or heat exposure therapy, including the release of cold shock proteins and/or heat shock proteins, respectively. Cold shock proteins can assist with the stabilization of RNA molecules, regulation of genes involved in the cold response, and cellular protection, to name a few. These cold shock proteins can enhance recovery and performance, improve immune function, provide neuroprotection and cognitive benefits, provide a metabolic boost, promote better weight management, enhance cellular longevity and cellular health, etc.

Heat exposure therapy, on the other hand, can enhance cardiovascular health and can be used to treat autoimmune disease, chronic pain, and fatigue conditions. Sauna therapy, for example, can also enhance mental health, exercise performance, metabolism, and detoxification as toxins are excreted from the body through the skin. Heat exposure therapy for about 10 minutes to about 45 minutes, or more typically from about 15 minutes to about 30 minutes, at a frequency of about 2 to 5 times per week, more typically from about 3 to 4 times per week, may significantly reduce all-cause mortality in humans. Saunas can be in the form of the traditional Finnish sauna, or it can be in the form of an infrared sauna, e.g., infrared, near infrared, far infrared, etc. Many saunas also include the ability hardware installed that allows the combination of full body heat therapy with red-light therapy. Temperature ranges can vary, with higher temperatures being used with the traditional Finnish sauna (which relies on heated rocks or other heated media to heat the space), e.g., from about 70° C. to about 105° C., but more typically from about 71° C. to about 93° C. Lower temperatures can be used for longer periods in some instances. Infrared saunas, on the other hand, can be used effectively at lower temperatures due to the penetration of the infrared energy into the skin. Typical temperature ranges can be from about 45° C. to about 75° C., though temperatures outside of these ranges can likewise be used. Heat exposure, combined with methylene blue and one or more of the secondary physiologically active compounds can enhance the health impact of the subject for many of the same reasons described elsewhere herein, including that related to red-light therapy protocols in particular.

It is noted that the terms “cold exposure therapy” and “heat exposure therapy” do not include the localized use of an ice pack or a heating pack often used to treat sore muscles and/or other types of pain. Typically, these therapies are full body (or nearly full body) therapies that exposes from about 75% to 100% of the subject by surface area to the cold or heat exposure therapy, e.g., cold plunge therapy usually involves the head and neck (and sometimes shoulders) remaining above the water surface. With that said, to the extent that an exposure therapy results in the release of shock proteins, it may be more localized than a full body (or 75%+ body) treatment. For example, exposing the face to icy cold water can simulate much of the effect of a cold plunge.

Cold Exposure Therapies with Co-administration of Methylthioninium Salt and Secondary Physiological Compound(s)

Mitochondrial enhancement can be achieved using cold plunge or other similar full body cold therapies, e.g., cryotherapy, and can be combined with methylthioninium salts and a secondary physiologically active compound(s) for enhanced or synergistic effect as well. When using cold plunge therapy to generate systemic release of cold shock proteins, example therapy times may range from about 1 minute to about 10 minutes, from about 2 minutes to about 10 minutes, or from about 2 minutes to about 8 minutes, depending on the temperature and any health concerns. Typically, from about 3 minutes to about 5 minutes is sufficient for cold exposure, though it may vary from subject to subject. The temperature of a cold plunge body of water can typically range from about 3° C. to about 16° C., though most typically, the temperature range can be from about 7° C. to about 15° C., or from about 7° C. to about 13° C. When using cryotherapy for the cold exposure therapy, example therapy times can be from about 1 minute to about 5 minutes, from about 1 minute to about 4 minutes, from about 1 minute to about 3 minutes, from about 2 minutes to about 4 minutes, or from about 2 minutes to about 3 minutes. A temperature range for the cryotherapy may be from about −120° C. to about −200° C., from about −125° C. to about −185° C., from about −130° C. to about −165° C., or from about −140° C. to about −200° C., for example.

To illustrate, cold exposure therapy can stimulate mitochondrial function and stimulate mitochondrial biogenesis. For a specific example, cold plunge therapy can be administered to a subject as described above in combination with the co-administration of compounds as described herein. Methylene blue can act as an alternative electron acceptor in the mitochondrial electron transport chain (ETC), potentially increasing ATP production. Notably, methylene blue can act as either an electron acceptor or an electron donor, depending on its form within the body of the subject. Regarding the secondary physiologically active compound, green tea (polyphenols) can stimulate biogenesis, urolithin A can enhance mitophagy (removal of damaged mitochondria) and improve mitochondrial function, guarana can generate new mitochondria and activate AMPK to stimulate metabolism, and pyrroloquinoline quinone (PQQ) can enhance mitochondrial biogenesis and function. Other secondary compounds that can be used for enhancing mitochondrial function include N-acetyl cysteine (NAC), glycine, vitamin B3, e.g., niacinamide, vitamin C, among others.

These combination therapies (including cold exposure therapy) can also be used for their antioxidant and/or anti-inflammatory properties. The temperatures and durations of use are set forth in detail above, and can be used within those parameters, for example. Methylene blue can provide the benefits as describe with respect to its antioxidant and anti-inflammatory properties as described herein. Regarding the secondary physiologically active compound, vitamin C, green tea (polyphenols), and/or guarana, for example, can be used in combination with methylene blue and cold exposure therapy for their antioxidant properties, including as the properties related to cellular mitochondria health. Other secondary compounds that can be used for enhancing antioxidant and/or anti-inflammation may include other compounds listed in Table 1, for example.

Other benefits of cold exposure or other full body cold therapy combined with the co-administration of the methylthioninium salt and the secondary physiologically active compound(s) may include enhanced metabolic activity, neuroprotection, improved energy and exercise recovery, anti-aging, and others. For example, cold exposure paired with methylene blue and guarana can enhance metabolic activity by enhancing insulin sensitivity and reducing insulin resistance. Cold exposure therapy paired with methylene blue and pyrroloquinoline quinone (PQQ) can increase BDNF levels to support cognitive function, provide neuroprotection for subjects with neurodegenerative diseases, and reduce inflammation/improve mitochondrial function in the brain. As another example, cold exposure therapy paired with methylene blue and guarana can ease sore muscles, reduce inflammation after workouts, enhance cellular energy production, and increase energy levels. Cold exposure therapy paired with methylene blue and urolithin A and/or guarana can have anti-aging effects due to the stimulation of the production of cold shock proteins that can assist with repairing damaged DNA, and both urolithin A and guarana both promote anti-aging properties.

### C. Oxygenation Therapies

In accordance with the present disclosure, various oxygenation therapies can be utilized with good efficacy, including therapies such as hyperbaric oxygen therapy, enhanced oxygen therapy, or the like. Enhanced oxygen therapy, for example, does not utilize an oxygen rich chamber under increased pressure, but rather simply adds additional oxygen to the air that the subject breaths, such as by the use of a cannula or mask connected via a supply hose to an oxygen supply or an enhanced oxygen supply. Hyperbaric oxygen therapy (HBOT), on the other hand, provides a subject with a high pressure, oxygen-rich environment that essentially forces higher concentrations of oxygen directly into the blood. This can provide conditions in which areas or tissues of the body that may have been oxygen deprived can receive more oxygen, and in some instances, can regenerate blood vessels and ultimately tissue to which blood delivery of oxygen may be increased.

Hyperbaric oxygen therapy chambers can be used at pressures greater than about 1 Atmospheres Absolute (ATA) up to about 4 ATA, depending on the type of chamber selected for use. ATA is used herein as a pressure standard, and includes the pressure of the system as a whole, including the local atmospheric pressure. Thus, 1 ATA is approximately the same as 1 standard atmosphere, but within an enclosed hyperbaric pressure chamber, than the local environmental pressure. The use of ATA in hyperbaric pressure chambers provides a convenient way to establishing interior chamber pressure that is uniform from location to location. In accordance with this, the pressure of the hyperbaric pressure chambers used in the present disclosure can typically range from about 1.25 ATA to about 4 ATA, from about 1.25 ATA to about 3 ATA, or from about 1.25 ATA to about 2.5 ATA, from about 1.5 ATA to about 4 ATA, or from about 1.5 ATA to about 3 ATA, for example.

In further detail, some hyperbaric pressure chambers having higher possible pressure limits compared to others. For example, there are at least two categories of hyperbaric chambers that are currently available for use, namely soft chambers and hard chambers. Soft hyperbaric chambers use flexible materials to generate the increased pressure therein, and can achieve pressures greater than the ambient pressure up to about 2 ATA, but more typically up to about 1.5 ATA of pressure. These tend to be more accessible, user-friendly, and portable for home or clinic use. Hard hyperbaric chambers, on the other hand, are constructed of rigid materials, e.g., stainless steel that can withstand higher pressures. An example hard hyperbaric chamber may be suitable for generating an oxygen-rich environment at pressures up to about 4 ATA, with a more typical range of up to about 3 ATA, e.g. from about 1.5 ATA to about 3 ATA. Hard hyperbaric chambers are more typically found in clinics, but are also available for personal ownership for subjects seeking higher pressure options to deliver higher concentrations of oxygen.

The chamber oxygen content of the atmosphere within the hyperbaric chamber can be at least about 99 vol % oxygen, e.g., 100 vol % oxygen, but lower concentrations of oxygen can be used with other gases normally present in the air, e.g., at least about 75% oxygen atmosphere. Typical treatment time that a subject may reside in the hyperbaric chamber can depend on the hyperbaric chamber pressure and the condition(s) being treated, but is typically in the range of from about 30 minutes to about 3 hours, from about 45 minutes to about 2.5 hours, or from about 1 hour to about 2 hours. In some examples, it is noted that HBOT can be used with other physiological therapies to improve blood flow, e.g., HBOT combined with red-light therapy can be used to improve cerebral blood flow and reduce oxidative stress.

Combining HBOT with the co-administration of methylthioninium salts and one or more of the secondary physiologically active compounds described herein can provide additive or even synergistic benefits, e.g., HBOT can be used in connection with the coadministration of methylene blue along with urolithin A and/or pyrroloquinoline quinone (PQQ) to enhance cellular oxygen utilization and improve mitochondrial function and overall health. Other additive benefits or synergies where HBOT would be beneficial can be realized by the coadministration of methylene blue with one or more of guarana, L-theanine, caffeine, green tea, vitamin C, and/or mineral oxychloride, among others. For example, methylene blue can enhance the effects of HBOT by improving cellular oxygen utilization, vitamin C can enhance the absorption and effectiveness of the methylene blue, and the combination of a stimulating and calming compound, e.g., caffeine and L-theanine, can provide balanced energy without causing jitters. In some examples, HBOT can be used to improve cognitive function (reduction in brain inflammation, growth of new brain cells, increase in oxygen supply to brain, treatment of traumatic brain injuries or stroke, enhancement in mental clarity), brain metabolism (such as in Alzheimer's and mild cognitive impairment patients), cellular repair, cerebral blood flow and oxygen supply to brain tissue, oxidative stress, inflammation and pain (decreasing inflammatory markers and swelling), energy production and metabolism, mood/mental state, physical performance, focus, organ function (including the brain), and/or neuroplasticity, to name a few. HBOT can also contribute to accelerated wound healing, e.g., chronic wounds, diabetic ulcers, etc., enhanced collagen production, boosted natural healing, increased cellular oxygen by raising the tissue-cellular diffusion gradient, etc.

An example protocol can include, for example, subjecting a subject to HBOT from 1 to 7 times per week, from 2 to 5 times per week, or from 3 to 5 times per week. Pressures can range from greater than ambient pressure, e.g., 1.25 ATA, up to about 4 ATA, from about 1.25 ATA to about 1.5 ATA, from about 1.5 ATA to about 3 ATA, or from about 1.25 ATA to about 2.5 ATA, for example. The chamber oxygen content can be greater than about 75 vol %, greater than about 90 vol %, or greater than about 99 vol %. Along with the HBOT, the subject can be orally administered a methylthioninium salt, such as methylene blue, along with guarana, green tea, L-theanine, or a combination thereof. Alternatively (or additionally), the subject can be administered methylene blue along with oxygenated minerals, vitamin C, or a combination thereof. Other secondary physiologically active compounds can likewise be used, such as those listed by way of example in Table 1 herein.

### Example Embodiments

In accordance with the disclosure herein, the following examples are illustrative of several embodiments of the present technology.
1. A physiological protection or enhancement system, comprising:
a methylthioninium salt-containing composition, comprising a methylthioninium salt and a secondary physiologically active compound selected from urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or a combination thereof; and
a physiological therapy device for use in combination with the methylthioninium salt-containing composition, wherein the physiological therapy device is selected from a thermal therapy device, an electromagnetic therapy device, an oxygenation therapy device, or a combination thereof.
2. The physiological protection or enhancement system of example 1, wherein the physiological therapy device includes the thermal therapy device and is selected from a cold plunge therapy device, a cryotherapy device, a sauna therapy device, a localized heat or cold therapy device, or a combination thereof.
3. The physiological protection or enhancement system of one of examples 1 to 2, wherein the physiological therapy device includes the electromagnetic therapy device and is selected from a red-light therapy device, a pulse electromagnetic field therapy device, an electric stimulation therapy device, or a combination thereof.
4. The physiological protection or enhancement system of one of examples 1 to 3, wherein the physiological therapy device includes the oxygenation therapy device and is selected from a hyperbaric oxygen therapy device, an enhanced oxygen therapy device, or a combination thereof.
5. The physiological protection or enhancement system of one of examples 1 to 4, wherein the methylthioninium salt-containing composition is in an oral dosage form selected from tablet, capsule, soft-gel, powder, liquid suspension or solution, or transmucosal oral formulation.
6. The physiological protection or enhancement system of one of examples 1 to 5, wherein the methylthioninium salt is in the form of methylthioninium chloride.
7. The physiological protection or enhancement system of one of examples 1 to 6, wherein the secondary physiologically active compound includes:
the urolithin A at a methylthioninium salt to urolithin A weight ratio from about 1:600 to about 1:4,
the pyrroloquinoline quinone at a methylthioninium salt to pyrroloquinoline quinone weight ratio from about 1:20 about 50:1,
the tesofensine at a methylthioninium salt to tesofensine weight ratio from about 1:2 to about 100:1, or
a combination thereof.
8. The physiological protection or enhancement system of example 7, wherein the methylthioninium salt-containing composition includes both the urolithin A and the pyrroloquinoline quinone.
9. The physiological protection or enhancement system of example 7, wherein the methylthioninium salt-containing composition includes the tesofensine and one or both of the urolithin A or the pyrroloquinoline quinone.
10. The physiological protection or enhancement system of example 7, wherein a single daily dose or divided daily dose of the methylthioninium salt-containing composition includes from about 1 mg to about 50 mg of the methylthioninium salt, and wherein the secondary physiologically active compound is selected from about 100 mg to about 1200 mg of the urolithin A, from about 0.5 mg to about 40 mg of the pyrroloquinoline quinone, from about 0.25 mg to about 4 mg of the tesofensine, or a combination thereof.
11. The physiological protection or enhancement system of example 7, wherein the secondary physiologically active compound includes the urolithin A, the pyrroloquinoline quinone, and at least one of the guarana, the L-theanine, the green tea, the caffeine, the mineral oxides and/or mineral chlorides, the N-acetylcysteine, the vitamin B3, the glycine, the vitamin C, or a combination thereof.
12. The physiological protection or enhancement system of example 11, wherein the physiological therapy device is the electromagnetic therapy device and includes a red-light therapy device, a pulsed electromagnetic field therapy device, or a combination thereof.
13. The physiological protection or enhancement system of example 11, wherein the physiological therapy device is the thermal therapy device and includes a cold plunge device, a cryochamber, or a combination thereof.
14. The physiological protection or enhancement system of example 7, comprising the nicotinamide adenine dinucleotide in the form of NADH, NAD+, or a combination thereof at a methylthioninium salt and nicotinamide adenine dinucleotide weight ratio from about 1:100 to about 5:2.
15. The physiological protection or enhancement system of example 7, wherein the physiological therapy device is selected from a red-light therapy device, a pulse electromagnetic field therapy device, a hyperbaric oxygen therapy device, a cold plunge device, or a combination thereof.
16. The physiological protection or enhancement system of one of examples 1 to 15, wherein the secondary physiologically active compound includes:
the caffeine at a methylthioninium salt to caffeine weight ratio from about 1:75 to about 1:4,
the L-theanine at a methylthioninium salt and the L-theanine weight ratio from about 1:200 to about 1:10, or
a combination thereof.
17. The physiological protection or enhancement system of example 16, comprising both the caffeine and the L-theanine.
18. The physiological protection or enhancement system of example 16, wherein the secondary physiologically active compound includes:
the green tea at a methylthioninium salt to green tea weight ratio from about 1:3 to about 1:25,
the guarana at a methylthioninium salt to guarana weight ratio of about 1:10 to about 1:40, or
a combination thereof,
wherein the green tea, the guarana, or both are a source for at least a portion of the caffeine present in the stress or anxiety reducing compound.
19. The physiological protection or enhancement system of example 16, wherein the secondary physiologically active compound includes the cannabidiol, the vitamin B6, the vitamin E, the manganese, the nicotine, or a combination thereof.
20. The physiological protection or enhancement system of example 16, wherein a single daily dose or divided daily dose of the methylthioninium salt-containing composition includes from about 1 mg to about 50 mg of methylthioninium salt, and wherein the secondary physiologically active compound is selected from about 20 mg to about 450 mg of caffeine, from about 25 mg to about 900 mg of L-theanine, or a combination thereof.
21. The physiological protection or enhancement system of example 16, wherein the physiological therapy device is selected from a red-light therapy device, a pulse electromagnetic field therapy device, a hyperbaric oxygen therapy device, a cold plunge device, or a combination thereof.
22. The physiological protection or enhancement system of one of examples 1 to 21, wherein the methylthioninium salt-containing composition is in the form of a liquid suspension, and wherein the secondary physiologically active compound includes the mineral blend of salts, oxides, and/or chlorides at a methylthioninium salt to mineral blend of salts, oxides, and/or chlorides weight ratio of about 100:1 to about 1:4, and wherein the methylthioninium salt-containing composition further includes hydrogen peroxide formed in situ.
23. The physiological protection or enhancement system of example 22, wherein the liquid suspension further comprises colloidal silver.
24. The physiological protection or enhancement system of example 23, wherein the colloidal silver is present at from about 5 ppm to about 50 ppm by weight and has an average particle size from about 3 nm to about 20 nm.
25. The physiological protection or enhancement system of example 22, wherein the weight ratio of the methylthioninium salt to the mineral blend of salts, oxides, and/or chlorides is from about 40:1 to about 2:1.
26. The physiological protection or enhancement system of example 22, wherein a single daily dose or a divided daily dose of the liquid suspension has a total volume from about 0.25 mL to about 5 mL, and the total volume of the liquid suspension provides includes from about 1 mg to about 50 mg of the methylthioninium salt and from about 0.2 mg to about 20 mg of the mineral blend of salts, oxides, and/or chlorides.
27. The physiological protection or enhancement system of example 22, wherein the liquid suspension further includes magnesium chloride, potassium chlorate, sodium chloride, magnesium oxide, or a combination thereof.
28. The physiological protection or enhancement system of example 22, wherein the liquid suspension further includes the cannabidiol, the vitamin B6, the vitamin E, the manganese, the nicotine, the caffeine, the L-theanine, or a combination thereof.
29. The physiological protection or enhancement system of example 22, wherein the physiological therapy device is selected from a red-light therapy device, a pulse electromagnetic field therapy device, a hyperbaric oxygen therapy device, a cold plunge device, or a combination thereof.
30. The physiological protection or enhancement system of one of examples 1 to 29, wherein the secondary physiologically active compound includes the vitamin C at a methylthioninium salt to vitamin C weight ratio from about 1:2 to about 1:10.
31. The physiological protection or enhancement system of example 30, wherein a single daily dose or divided daily dose of the methylthioninium salt-containing composition includes from about 1 mg to about 50 mg of methylthioninium salt, and wherein the secondary physiologically active compound includes from about 50 mg to about 1000 mg of the vitamin C.
32. The physiological protection or enhancement system of example 30, wherein the secondary physiologically active compound further includes the cannabidiol, the vitamin B6, the vitamin E, the manganese, the nicotine, the caffeine, the L-theanine, or a combination thereof.
33. The physiological protection or enhancement system of example 30, wherein the physiological therapy device is selected from a red-light therapy device, a pulse electromagnetic field therapy device, a hyperbaric oxygen therapy device, a cold plunge device, or a combination thereof.
34. The physiological protection or enhancement system of one of examples 1 to 33, wherein the secondary physiologically active compound includes:
the glycine at a methylthioninium salt to glycine weight ratio from about 1:3 to about 1:25,
the N-acetylcysteine at a methylthioninium salt to N-acetylcysteine weight ratio from about 1:3 to about 1:25,
the vitamin B3 at a methylthioninium salt to vitamin B3 weight ratio from about 1:2 to about 1:12, or
a combination thereof.
35. The physiological protection or enhancement system of example 34, wherein the glycine and the N-acetylcysteine are both present at a weight ratio from about 1:2 to about 2:1.
36. The physiological protection or enhancement system of example 34, wherein the vitamin B3 is in the form of niacinamide.
37. The physiological protection or enhancement system of example 34, wherein a single daily dose or divided daily dose of the methylthioninium salt-containing composition includes from about 1 mg to about 50 mg of methylthioninium salt, and wherein the secondary physiologically active compound includes from about 20 mg to about 100 mg of the glycine, from about 25 mg to about 100 mg of the N-acetylcysteine, from about 5 mg to about 50 mg of the vitamin B3, or a combination thereof.
38. The physiological protection or enhancement system of example 34, wherein the secondary physiologically active compound further includes the cannabidiol, the vitamin B6, the vitamin E, the manganese, the nicotine, the caffeine, the L-theanine, or a combination thereof.
39. The physiological protection or enhancement system of example 34, wherein the physiological therapy device is selected from a red-light therapy device, a pulse electromagnetic field therapy device, a hyperbaric oxygen therapy device, a cold plunge device, or a combination thereof.
40. The physiological protection or enhancement system of one of examples 1 to 39, wherein the secondary physiologically active compound includes:
the cannabinoid at a methylthioninium salt to cannabinoid weight ratio from about 1:3 to about 1:10,
the vitamin B6 at a methylthioninium salt to vitamin B6 weight ratio from about 10:1 to about 2:1,
the vitamin E at a methylthioninium salt to vitamin E weight ratio from about 1:3 to about 3:1,
the manganese at a methylthioninium salt to manganese metal content weight ratio from about 10:1 to about 2:1, or
a combination thereof.
41. The physiological protection or enhancement system of example 40, comprising at least two of the cannabinoid, the vitamin B6, the vitamin E, or the manganese.
42. The physiological protection or enhancement system of example 40, wherein the caffeine and the vitamin E are present at a weight ratio from about 1:2 to about 1:1, the caffeine and the manganese metal content are present at a weight ratio from about 7:1 to about 2.5:1, or both.
43. The physiological protection or enhancement system of example 40, wherein a single daily dose or divided daily dose of the methylthioninium salt-containing composition includes from about 1 mg to about 50 mg of methylthioninium salt, and wherein the secondary physiologically active compound includes from about 10 mg to about 100 mg of the cannabinoid, from about 0.2 mg to about 6 mg of the vitamin B6, about 3 mg to about 30 mg of the vitamin E, about 0.5 mg to about 8 mg of the manganese metal content, or a combination thereof.
44. The methylthioninium salt-containing composition of example 40, comprising the nicotine, the caffeine, the L-theanine, or a combination thereof.
45. The physiological protection or enhancement system of example 40, wherein the secondary physiologically active compound further includes one or more of the nicotine, the caffeine, the L-theanine, or a combination thereof.
46. The physiological protection or enhancement system of one of examples 1 to 45, further comprising black pepper at a sufficient concentration to enhance absorption of the methylthioninium salt, the secondary physiologically active compound, or both.
47. The physiological protection or enhancement system of example 40, wherein the physiological therapy device is selected from a red-light therapy device, a pulse electromagnetic field therapy device, a hyperbaric oxygen therapy device, a cold plunge device, or a combination thereof.
48. The physiological protection or enhancement system of one of examples 1 to 47 wherein the physiological therapy device includes a red-light therapy device adapted to delivery red-light therapy to a surface of a subject at a wavelength from about 600 nm to about 750 nm, from greater than about 750 nm to about 1400 nm, or a combination thereof.
49. The physiological protection or enhancement system of one of examples 1 to 47, wherein the physiological therapy device includes a pulsed electromagnetic field device adapted to delivery pulsed electromagnetic field therapy at a pulse repetition frequency from greater than 0 Hz to about 50 Hz at a radiation intensity from about 10 milligauss to about 1000 gauss.
50. The physiological protection or enhancement system of one of examples 1 to 47, wherein the physiological therapy device includes a hyperbaric oxygen therapy device adapted to delivery oxygen from a pressurized oxygen-rich atmosphere to a subject at a pressure from about 1.25 ATA to about 4 ATA, wherein the oxygen-rich atmosphere includes from about 75 vol % oxygen to 100 vol % oxygen.
51. A method of protecting or enhancing a physiological function, comprising:
orally co-administering to a methylthioninium salt and a secondary physiologically active compound selected from urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or a combination thereof to a subject; and
subjecting the subject to a physiological therapy selected from thermal therapy, electromagnetic therapy, oxygenation therapy, or a combination thereof while the subject is dosed with clinically effective blood serum levels the methylthioninium salt and the secondary physiologically active compound.
52. The method of example 51, wherein the physiological therapy includes the electromagnetic therapy and is selected from red-light therapy, pulse electromagnetic field therapy, electric stimulation therapy, or a combination thereof.
53. The method of example 52, wherein the physiological therapy includes the red-light therapy conducted for from about 5 minutes to about 60 minutes at a peak wavelength:
from about 600 nm to about 750 nm,
from greater than about 750 nm to about 1400 nm,
or a combination thereof.
54. The method of example 52, wherein the physiological therapy includes the pulse electromagnetic field therapy conducted for from about 5 minutes to about 60 minutes at a pulse repetition frequency greater than 0 Hz to about 50 Hz at a radiation intensity from about 10 milligauss to about 1000 gauss.
55. The method of example 52, wherein the physiological therapy includes the pulse electromagnetic field therapy conducted for from about 5 minutes to about 60 minutes at a pulse repetition frequency from about 1 Hz to about 40 Hz at a radiation intensity from about 200 milligauss to about 2 gauss.
56. The method of example 52, wherein the physiological therapy includes the pulse electromagnetic field therapy conducted at a pulse repetition frequency including a Schumann resonance from about 7.6 Hz to about 8 Hz.
57. The method of one of examples 51 to 56 wherein the physiological therapy includes the oxygenation therapy and is selected from hyperbaric oxygen therapy, enhanced oxygen therapy, or a combination thereof.
58. The method of example 57, wherein the physiological therapy includes the hyperbaric oxygen therapy conducted for from about 15 minutes to about 2 hours in a hyperbaric chamber having a chamber pressure from greater than 1ATA to about 4 ATA with a chamber oxygen content from about 75 vol % to 100 vol %.
59. The method of one of examples 51 to 58, wherein the physiological therapy includes the thermal therapy and is selected from cold plunge therapy, cryotherapy, sauna therapy, localized heat or cold therapy, or a combination thereof.
60. The method of example 59, wherein the physiological therapy is the cold plunge therapy for about 1 minutes to 10 minutes at a temperature from about 3° C. to about 16° C.
61. The method of example 59, wherein the physiological therapy is the cryotherapy for about 1 minute to about 5 minutes at a temperature from about −120° C. to about −200° C.
62. The method of one of examples 51 to 61, wherein the methylthioninium salt and the secondary physiologically active compound are co-formulated together as a methylthioninium salt-containing composition.
63. The method of example 62, wherein the methylthioninium salt-containing composition is in an oral dosage form selected from tablet, capsule, soft-gel, powder, liquid suspension or solution, or transmucosal oral formulation.
64. The method of one of examples 51 to 63, wherein the methylthioninium salt is in the form of methylthioninium chloride.
65. The method of one of examples 51 to 64, further comprising co-administering black pepper at a sufficient concentration to enhance absorption of the methylthioninium salt, the secondary physiologically active compound, or both.
66. The method of one of examples 51 to 65, wherein the secondary physiologically active compound includes:
the urolithin A at a methylthioninium salt to urolithin A weight ratio from about 1:600 to about 1:4,
the pyrroloquinoline quinone at a methylthioninium salt to pyrroloquinoline quinone weight ratio from about 1:20 to about 50:1,
the tesofensine at a methylthioninium salt to tesofensine weight ratio from about 1:2 to about 100:1, or
a combination thereof.
67. The method of example 66, wherein orally co-administering further includes orally co-administering nicotinamide adenine dinucleotide in the form of NADH, NAD+, or a combination thereof at a daily dosage from about 20 mg to about 200 mg.
68. The method of example 66, wherein the methylthioninium salt is dosed on a daily basis at a sufficient concentration to stimulate ATP production and the urolithin A is dosed at a sufficient concentration to recognize or repair mitochondria damage.
69. The method of example 66, wherein the methylthioninium salt is dosed on a daily basis at a sufficient concentration to stimulate ATP production and the pyrroloquinoline quinone is dosed at a sufficient concentration to enhance nicotinamide adenine dinucleotide production, mitochondrial mitophagy, or a combination thereof.
70. The method of example 66, wherein the methylthioninium salt is dosed on a daily basis at a sufficient concentration to stimulate ATP production and the tesofensine is dosed at a sufficient concentration to enhance cognition due to increased levels of dopamine, norepinephrine, serotonin, or a combination thereof in the brain, feelings of fullness and satisfaction by curbing appetite, or a combination thereof.
71. The method of example 66, wherein the methylthioninium salt is dosed on a daily basis at about 1 mg to about 50 mg per day, and the secondary physiologically active compound is dosed on a daily basis at from about 100 mg to about 1200 mg of the urolithin A, from about 0.5 mg to about 40 mg of the pyrroloquinoline quinone, from about 0.25 mg to about 4 mg of the tesofensine, or a combination thereof.
72. The method of example 66, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
73. The method of one of examples 51 to 72, wherein the secondary physiologically active compound is selected from caffeine at a methylthioninium chloride to caffeine weight ratio from about 1:75 to about 1:4, L-theanine at a methylthioninium chloride to L-theanine weight ratio from about 1:200 to about 1:10, L-theanine, or a combination thereof.
74. The method of example 73, wherein both the caffeine and the L-theanine orally co-administered with the methylthioninium salt at a caffeine to L-theanine weight ratio from about 1:5 to about 1:1.
75. The method of example 73, comprising guarana, green tea, or a combination thereof as a source of the caffeine.
76. The method of example 73, wherein the methylthioninium salt and the secondary physiologically active compound are dosed on a daily basis at sufficient concentrations to treat attention disorders selected from attention deficit hyperactivity disorder (ADHD) or attention deficit disorder (ADD), to enhance or sustain weight loss, to enhance metabolism of glucagon-like peptide-1 (GLP-1).
77. The method of example 73, wherein the methylthioninium salt is dosed on a daily basis at about 1 mg to about 50 mg per day, and the secondary physiologically active compound is dosed on a daily basis at from about 20 mg to about 450 mg of caffeine, from about 25 mg to about 900 mg of L-theanine, or a combination thereof.
78. The method of example 73, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
79. The method of one of examples 51 to 78, wherein the secondary physiologically active compound is selected from a liquid suspension of mineral blend of salts, oxides, and/or chlorides having a methylthioninium salt to mineral blend of salts, oxides, and/or chlorides weight ratio of about 100:1 to about 1:4 and hydrogen peroxide formed in situ, wherein the liquid suspension is either co-formulated with the methylthioninium salt or is orally co-administered as a separate composition.
80. The method of example 79, wherein the liquid suspension further comprises colloidal silver having an average particle size from about 3 nm to about nm, magnesium chloride, or a combination thereof.
81. The method of example 79, wherein the methylthioninium salt and the secondary physiologically active compound are dosed on a daily basis at sufficient concentrations to treat long COVID or vaccine-induced spike protein damage, brain fog, dementia, Lyme disease, chronic fatigue, or a combination thereof.
82. The method of example 79, wherein the methylthioninium salt is dosed on a daily basis at about 1 mg to about 50 mg per day, and the secondary physiologically active compound is dosed on a daily basis at from about 0.2 mg to about 20 mg of the mineral blend of salts, oxides, and/or chlorides.
83. The method of example 79, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
84. The method of one of examples 51 to 83, wherein the secondary physiologically active compound includes vitamin C at a methylthioninium salt to vitamin C weight ratio from about 1:2 o about 1:10.
85. The method of example 84, further comprising the cannabidiol, the vitamin B6, the vitamin E, the manganese, the nicotine, the caffeine, the L-theanine, GLP-1 enhancement medication, or a combination thereof.
86. The method of example 84, wherein the methylthioninium salt is dosed on a daily basis at about 1 mg to about 50 mg per day, and the secondary physiologically active compound is dosed on a daily basis at from about 50 mg to about 1000 mg of the vitamin C.
87. The method of example 84, wherein the methylthioninium salt and the secondary physiologically active compound are dosed on a daily basis at sufficient concentrations to treat early-stage cognitive decline associated with dementia or Alzheimer's disease, support weight loss support.
88. The method of example 84, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
89. The method of one of examples 51 to 88, wherein the secondary physiologically active compound includes glycine at a methylthioninium salt to glycine weight ratio from about 1:3 to about 1:25, N-acetylcysteine at a methylthioninium salt to N-acetylcysteine weight ratio from about 1:3 to about 1:25, vitamin B3 at a methylthioninium salt to vitamin B3 weight ratio from about 1:2 to about 1:12, or a combination thereof.
90. The method of example 89, wherein the glycine and the N-acetylcysteine are both present at a weight ratio from about 1:2 to about 2:1.
91. The method of example 89, wherein the vitamin B3 is in the form of niacinamide, nicotinic acid, or a combination thereof.
92. The method of example 89, wherein the methylthioninium salt and the secondary physiologically active compound are dosed on a daily basis at sufficient concentrations to treat general cognitive decline, traumatic brain injury, or a combination thereof.
93. The method of example 89, wherein the methylthioninium salt is dosed on a daily basis at about 1 mg to about 50 mg per day, and the secondary physiologically active compound is dosed on a daily basis at from about at from about 20 mg to about 100 mg of the glycine, from about 25 mg to about 100 mg of the N-acetylcysteine, from about 5 mg to about 50 mg of the vitamin B3, or a combination thereof.
94. The method of example 84, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
95. The method of one of examples 51 to 94, wherein the secondary physiologically active compound includes the cannabinoid at a methylthioninium salt to cannabinoid weight ratio from about 1:3 to about 1:10, the vitamin B6 at a methylthioninium salt to vitamin B6 weight ratio from about 10:1 to about 2:1, the vitamin E at a methylthioninium salt to vitamin E weight ratio from about 1:3 to about 3:1, the manganese at a methylthioninium salt to manganese metal content weight ratio from about 10:1 to about 2:1, or a combination thereof.
96. The method of example 95, wherein the caffeine and the vitamin E are present at a weight ratio from about 1:2 to about 1:1, the caffeine and the manganese metal content are present at a weight ratio from about 7:1 to about 2.5:1, or both.
97. The method of example 95, wherein the methylthioninium salt and the secondary physiologically active compound are dosed on a daily basis at sufficient concentrations to treat seizures, epilepsy, sleep disorders, or a combination thereof.
98. The method of example 95, wherein the methylthioninium salt is dosed on a daily basis at about 1 mg to about 50 mg per day, and the secondary physiologically active compound is dosed on a daily basis at from about from about 10 mg to about 100 mg of the cannabinoid, from about 0.2 mg to about 6 mg of the vitamin B6, about 3 mg to about 30 mg of the vitamin E, about 0.5 mg to about 8 mg of the manganese metal content, or a combination thereof.
99. The method of example 95, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
100. A physiological enhancement system, comprising:
a liquid nutritional supplement composition including from about 80 wt % to about 99 wt % liquid carrier (or water); from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and from about 0.1 wt % to about 5 wt % methylated vitamin B12, from about 0.1 wt % to about 5 wt % vitamin C, or both; and
a physiological therapy device for use in combination with the liquid nutritional supplement composition, wherein the physiological therapy device is selected from a thermal therapy device, an electromagnetic therapy device, an oxygenation therapy device, or a combination thereof.
101. The physiological enhancement system of example 100, further comprising a secondary physiologically active compound having enhanced bioavailability in the presence of the mineral blend.
102. The physiological enhancement system of example 101, wherein the secondary physiologically active compound includes a member selected from urolithin A, pyrroloquinoline quinone, tesofensine, nicotinamide adenine dinucleotide, caffeine, L-theanine, green tea, guarana, colloidal silver, glycine, N-acetylcysteine, vitamin B3, cannabidiol, vitamin B6, vitamin E, manganese, nicotine, GLP-1 agonist, or a combination thereof.
103. The physiological enhancement system of example 100, further comprising a methylthioninium salt as part of the liquid nutritional supplement or as a separate dosage form.
104. The physiological enhancement system of example 103, wherein the methylthioninium salt and the mineral blend are present at a weight ratio of about 100:1 to about 1:4.
105. The physiological enhancement system of example 100, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.
106. A method of protecting or enhancing a physiological function, comprising:
orally administering a liquid nutritional supplement composition to a subject, the liquid nutritional supplement composition including from about 80 wt % to about 99 wt % liquid carrier (or water); from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and from about 0.1 wt % to about 5 wt % methylated vitamin B12, from about 0.1 wt % to about 5 wt % vitamin C, or both; and
subjecting the subject to a physiological therapy selected from thermal therapy, electromagnetic therapy, oxygenation therapy, or a combination thereof while the subject is dosed with clinically effective levels of the liquid nutritional supplement composition to enhance the physiological function, or to enhance the absorption of methylthioninium salt and/or a secondary physiologically active compound to enhance the physiological function.
107. The method of example 106, further comprising administering a secondary physiologically active compound having enhanced bioavailability in the presence of the mineral blend.
108. The method of example 107, wherein the secondary physiologically active compound includes a member selected from urolithin A, pyrroloquinoline quinone, tesofensine, nicotinamide adenine dinucleotide, caffeine, L-theanine, green tea, guarana, colloidal silver, glycine, N-acetylcysteine, vitamin B3, cannabidiol, vitamin B6, vitamin E, manganese, nicotine, GLP-1 agonist, or a combination thereof.
109. The method of example 106, further comprising administering a methylthioninium salt as part of the liquid nutritional supplement or as a separate dosage form.
110. The method of example 109, wherein the methylthioninium salt and the mineral blend are present at a weight ratio of about 100:1 to about 1:4.
111. The method of example 106, wherein the physiological therapy includes:
the electromagnetic therapy selected from red-light therapy, pulse electromagnetic field therapy, or both,
the oxygenation therapy in the form of hyperbaric oxygen therapy,
the thermal therapy in the form of cold plunge therapy, cryotherapy, or both, or
a combination thereof.

### Definitions

In describing and claiming the present technology, the following terminology will be used.

The singular forms “a,” “an,” and “the” include plural references unless the context clearly dictates otherwise. Thus, for example, reference to “an additive” includes reference to one or more of such components, “a suspension” includes reference to one or more of such suspensions, and “the mixing” refers to one or more of such mixing steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, “about” refers to a degree of deviation based on experimental error typical for the particular property identified. The latitude provided by the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” expressly includes “exactly,” consistent with the discussion below regarding ranges and numerical data.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the terms “treatment” or “treating” of a condition and/or a disease in a mammal means preventing the condition or disease, that is, avoiding any clinical symptoms of the disease; inhibiting the condition or disease, that is, arresting the development or progression of clinical symptoms; and/or relieving the condition or disease, that is, causing the regression of clinical symptoms and/or healing. In further detail, “treating” can include oral administration of any of the methylthioninium salt-containing compounds described herein to heal a subject of a condition or disease, reduce or ameliorate symptoms of a condition or disease, prevent or protect a subject from experiencing or relapsing relative to a condition or disease, causing a condition or disease to regress or retreat, etc.

“Oral” routes of administration include oral ingestion (swallowing) via capsule, tablet, soft-gel, powder, or liquid, e.g., solution or suspension, and/or via transmucosal oral administration, e.g., buccal, sublabial, sublingual, etc. In some instances, some transmucosal oral delivery may occur prior to or after swallowing the bulk of the oral dosage form.

### EXAMPLES

The following examples illustrate embodiments of the disclosure that are presently best known. However, it is to be understood that the following are only exemplary or illustrative of the application of the principles of the present technology. Numerous modifications and alternative compositions, methods, and systems may be devised by those skilled in the art without departing from the spirit and scope of the present disclosure. The appended claims are intended to cover such modifications and arrangements. Thus, while the present disclosure has been described herein with particularity, the following examples provide further detail in connection with what are presently deemed to be practical embodiments of the disclosure.

The preparation of various methylthioninium salt-containing compositions are exemplified herein (see Examples 1-16 below, which are identified more generically as MB1-MB14, MB20, and MB23 in accordance with Table 1 and/or Table 3 above). Some of those compositions were evaluated in subjects suffering from various conditions. The use of various example physiological therapies, e.g., electromagnetic therapy, oxygenation therapy, or thermal therapy can be used in combination with these and other example coadministration regimens to enhance their effectiveness.

### Example 1—Preparation of Methylthioninium Salt-Containing Composition 1 (MB1)

A methylthioninium salt-containing composition (MB1) is prepared by admixing pulverized granules/powder of methylthioninium chloride, caffeine, and L-theanine at a 1:20:40 weight ratio. In further detail, other formulation excipients are included for purposes of forming tablets and/or a granule blend for loading into capsules. The granules are then compressed into tablets or loaded into capsules, with each tablet/capsule including about 2.5 mg methylthioninium chloride, 50 mg caffeine, and 100 mg L-theanine. In some examples, when forming compressed tablets, the tablets may be coated with a wax or polymer applied thereto. It is noted that the various components can be combined in liquid form and dried, or can be delivered as a liquid suspension.

### Example 2—Preparation of Methylthioninium Salt-Containing Composition 2 (MB2)

A methylthioninium salt-containing composition (MB2) is prepared by admixing pulverized granules or a powder of methylthioninium chloride, green tea, guarana, and L-theanine at a 1:10:11.4:40 weight ratio. In further detail, other formulation excipients are included for purposes of forming tablets and/or a granule blend for loading into capsules. The granules are then compressed into tablets or loaded into capsules, with each tablet/capsule including about 2.5 mg methylthioninium chloride, 25 mg of green tea (having 50 wt % caffeine), guarana (having 25 wt % caffeine), and 100 mg L-theanine. In some examples, when forming compressed tablets, the tablets may be coated with a wax or polymer applied thereto. It is noted that the various components can be combined in liquid form and dried, or can be delivered as a liquid suspension.

### Example 3—Preparation of Methylthioninium Salt-containing Composition 3 (MB3)

A methylthioninium salt-containing composition (MB3) is prepared by admixing pulverized granules or a powder of methylthioninium chloride and a mineral oxide/chloride suspension to form a liquid suspension. In this example, hydrogen peroxide is formed and can be present at about 10 wt % or slightly less, with mineral oxychlorides being detectable as an intermediate molecule during production of the hydrogen peroxide. The methylene blue is added to an aqueous carrier at a concentration of about 10 mg dosage per mL (in a 30 ml bottle). The mineral oxide/chloride suspension, which includes magnesium chloride, potassium chlorate, sodium chloride, and magnesium oxide, is added at about 1.5 vol %. This liquid suspension can be orally administered as a liquid, or can be placed in one or more soft gel capsules.

### Example 4—Preparation of Methylthioninium Salt-containing Composition 4 (MB4)

A methylthioninium salt-containing composition (MB4) is prepared by admixing methylthioninium chloride, a mineral oxide/chloride suspension, and colloidal silver to form a liquid suspension. In this example, hydrogen peroxide is formed and can be present at about 10 wt % or slightly less, with mineral oxychlorides being detectable as an intermediate molecule during production of the hydrogen peroxide. Essentially, the methylene blue is added to an aqueous carrier at a concentration of about 10 mg dosage per mL (in a 30 ml bottle). The mineral oxide/chloride suspension, which includes magnesium chloride, potassium chlorate, sodium chloride, magnesium oxide is added at about 1.5 vol %. The colloidal silver (Ag404) in this example is added to the liquid suspension to be present in the final composition at about 20 ppm (elemental silver particles having an average particle size of about 15 nm). This liquid suspension can be orally administered as a liquid, or can be placed in one or more soft gel capsules.

### Example 5-Preparation of Methylthioninium Salt-Containing Composition 5 (MB5)

A methylthioninium salt-containing composition (MB5) is prepared by admixing pulverized granules or a powder of methylthioninium chloride and vitamin C at about a 1:5 weight ratio. In further detail, other formulation excipients are included for purposes of forming tablets and/or a granule blend for loading into capsules. The granules are then compressed into tablets or loaded into capsules, with each tablet/capsule including about 15 mg methylthioninium chloride and about 75 mg of vitamin C. In some examples, when forming compressed tablets, the tablets may be coated with a wax or polymer applied thereto. It is noted that the various components can be combined in liquid form and dried, or can be delivered as a liquid suspension.

### Example 6-Preparation of Methylthioninium Salt-Containing Composition 6 (MB6)

A methylthioninium salt-containing composition (MB6) is prepared by admixing pulverized granules or a powder of methylthioninium chloride, glycine, N-acetylcysteine (NAC), and vitamin B3 (niacinamide) at a 1:10:10:5 weight ratio. In further detail, other formulation excipients are included for purposes of forming tablets and/or a granule blend for loading into capsules. The granules are then compressed into tablets or loaded into capsules, with each tablet/capsule including about 5 mg methylthioninium chloride, 50 mg glycine, 50 mg N-acetylcysteine, and 25 mg niacinamide. In some examples, when forming compressed tablets, the tablets may be coated with a wax or polymer applied thereto. It is noted that the various components can be combined in liquid form and dried, or can be delivered as a liquid suspension.

### Example 7-Preparation of Methylthioninium Salt-Containing Composition 7 (MB7)

A methylthioninium salt-containing composition (MB7) is prepared by admixing pulverized granules or a powder of methylthioninium chloride, a cannabinoid(s) (CBD), vitamin B6 (pyridoxal 5′ phosphate), vitamin E, and manganese (which can be included in the form of a citrate, gluconate, ascorbate, or amino acid chelate, for example) at a 2.5:25:1:7.5:1.5 weight ratio. The manganese content is based on the metal content. The cannabinoid is provided as dry particulate of a 20 wt % water soluble composition. Other formulation excipients are included for the purpose of forming tablets and/or a granule blend for loading into capsules. The admixture is then compressed into tablets or loaded into capsules, with each tablet/capsule including about 2.5 mg methylthioninium chloride, 25 mg of cannabinoid(s), 1 mg of vitamin B6, 7.5 mg of vitamin E, and 1.5 mg of manganese (by metal content). In some examples, when forming compressed tablets, the tablets may be coated with a wax or polymer applied thereto. It is noted that the various components can be combined in liquid form and dried, or can be delivered as a liquid suspension.

### Example 8-Preparation of Methylthioninium Salt-Containing Composition 8 (MB8)

Three methylthioninium salt-containing compositions (MB8) are prepared that include both a methylthioninium salt and urolithin A as the active compounds, as follows:
1. A methylthioninium salt-containing composition (MB8-A) is prepared by admixing pulverized granules/powder of methylthioninium chloride and urolithin A at about a 1:16.6 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 15 mg methylthioninium chloride and about 250 mg urolithin A. In some examples, when forming compressed tablets, the tablets may be coated with a wax or polymer applied thereto. It is noted that the various components can be combined in liquid form and dried, or can be delivered as a liquid suspension.
2. A methylthioninium salt-containing composition (MB8-B) is prepared by admixing pulverized granules/powder of methylthioninium chloride and urolithin A at about a 1:70 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride and about 350 mg urolithin A. In some examples, when forming compressed tablets, the tablets may be coated with a wax or polymer applied thereto. It is noted that the various components can be combined in liquid form and dried, or can be delivered as a liquid suspension.
3. A methylthioninium salt-containing composition (MB8-C) is prepared by admixing pulverized granules/powder of methylthioninium chloride and urolithin A at about a 1:50 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride and about 250 mg urolithin A. In some examples, when forming compressed tablets, the tablets may be coated with a wax or polymer applied thereto. It is noted that the various components can be combined in liquid form and dried, or can be delivered as a liquid suspension.

### Example 9-Preparation of Methylthioninium Salt-containing Composition 9 (MB9)

A methylthioninium salt-containing composition (MB9) is prepared by admixing pulverized granules/powder of methylthioninium chloride and pyrroloquinoline quinone (PQQ) at about a 1:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 10 mg methylthioninium chloride and about 10 mg of pyrroloquinoline quinone. In some examples, when forming compressed tablets, the tablets may be coated with a wax or polymer applied thereto. It is noted that the various components can be combined in liquid form and dried, or can be delivered as a liquid suspension.

### Example 10-Preparation of Methylthioninium Salt-Containing Composition 10 (MB10)

A methylthioninium salt-containing composition (MB10) is prepared by admixing pulverized granules/powder of methylthioninium chloride and tesofensine at about a 30:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 15 mg methylthioninium chloride and about 0.5 mg of tesofensine. A recommended daily dosage for this composition can be two tablets per day for a total of 10 mg methylthioninium chloride and 1 mg tesofensine. In some examples, when forming compressed tablets, the tablets may be coated with a wax or polymer applied thereto. It is noted that the various components can be combined in liquid form and dried, or can be delivered as a liquid suspension.

### Example 11-Preparation of Methylthioninium Salt-Containing Composition 11 (MB11)

A methylthioninium salt-containing composition (MB11) is prepared by admixing pulverized granules/powder of methylthioninium chloride and nicotinamide adenine dinucleotide at about a 1:10 weight ratio. In further detail, other formulation excipients are included for purposes of forming tablets and/or a granule blend for loading into capsules. The granules are then compressed into tablets (or loaded into capsules), with each oral dose, divided over 2 tablets/capsules, including about 15 mg methylthioninium chloride and about 150 mg of nicotinamide adenine dinucleotide. Thus, each tablet/capsule would have half (½) of the milligram content of the full oral dose. In some examples, when forming compressed tablets, the tablets may be coated with a wax or polymer applied thereto. It is noted that the various components can be combined in liquid form and dried, or can be delivered as a liquid suspension.

### Example 12-Preparation of Methylthioninium Salt-Containing Composition 12 (MB12)

A methylthioninium salt-containing composition (MB12) is prepared by admixing pulverized granules/powder of methylthioninium chloride, urolithin A, and nicotinamide adenine dinucleotide at about a 1:75:10 weight ratio. In further detail, other formulation excipients are included for purposes of forming tablets and/or a granule blend for loading into capsules. The granules are then compressed into tablets (or loaded into capsules), with each oral dose, divided over 2 tablets/capsules, including about 10 mg methylthioninium chloride, about 750 mg urolithin A, and about 100 mg of nicotinamide adenine dinucleotide. Thus, each tablet/capsule would have half (½) of the milligram content of the full oral dose. In some examples, when forming compressed tablets, the tablets may be coated with a wax or polymer applied thereto. It is noted that the various components can be combined in liquid form and dried, or can be delivered as a liquid suspension.

### Example 13-Preparation of Methylthioninium Salt-Containing Composition 13 (MB13)

A methylthioninium salt-containing composition (MB13) is prepared by admixing pulverized granules/powder of methylthioninium chloride, urolithin A, and pyrroloquinoline quinone at about a 1:25:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 250 mg urolithin A, about 10 mg methylthioninium chloride, and about 10 mg of pyrroloquinoline quinone. In some examples, when forming compressed tablets, the tablets may be coated with a wax or polymer applied thereto. It is noted that the various components can be combined in liquid form and dried, or can be delivered as a liquid suspension.

### Example 14-Preparation of Methylthioninium Salt-Containing Composition 14 (MB14)

A methylthioninium salt-containing composition (MB14) is prepared by admixing pulverized granules/powder of methylthioninium chloride, urolithin A, pyrroloquinoline quinone, and tesofensine at about a 10:300:4:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride, about 150 mg urolithin A, about 2 mg of pyrroloquinoline quinone, and about 0.5 mg of tesofensine. A recommended daily dosage for this composition can be one or two tablets per day. For one tablet, the total daily dosage for each compound can be 5 mg methylthioninium chloride, 150 mg of urolithin A, 2 mg of pyrroloquinoline quinone, and 0.5 mg tesofensine. For two tablets, the total daily dosage for each compound can be 10 mg methylthioninium chloride, 300 mg of urolithin A, 4 mg of pyrroloquinoline quinone, and 1 mg tesofensine. In some examples, when forming compressed tablets, the tablets may be coated with a wax or polymer applied thereto. It is noted that the various components can be combined in liquid form and dried, or can be delivered as a liquid suspension. In some examples, the methylthioninium salt containing composition (MB10) can be co-formulated to also include 250 mg of nicotinamide adenine dinucleotide per tablet.

### Example 15-Preparation of Methylthioninium Salt-Containing Composition 20 (MB20)

A methylthioninium salt-containing composition (MB20) is prepared by admixing pulverized granules/powder of methylthioninium chloride, urolithin A, nicotinamide adenine dinucleotide, and pyrroloquinoline quinone at about a 2:200:40:1 weight ratio. In further detail, other formulation excipients are included for purposes of forming tablets and/or a granule blend for loading into capsules. The granules are then compressed into tablets (or loaded into capsules), with each oral dose, divided over 2 tablets or capsules, including about 5 mg methylthioninium chloride, about 500 mg urolithin A, about 100 mg nicotinamide adenine dinucleotide, and about 2.5 mg of pyrroloquinoline quinone. Thus, each tablet/capsule would have half (½) of the milligram content of the full oral dose. In some examples, when forming compressed tablets, the tablets may be coated with a wax or polymer applied thereto. It is noted that the various components can be combined in liquid form and dried, or can be delivered as a liquid suspension.

### Example 16-Preparation of Methylthioninium Salt-Containing Composition 23 (MB23)

A methylthioninium salt-containing composition (MB23) is prepared by admixing pulverized granules/powder of methylthioninium chloride, pyrroloquinoline quinone, and tesofensine at about a 15:20:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 7.5 mg methylthioninium chloride, about 10 mg of pyrroloquinoline quinone, and about 0.5 mg of tesofensine. A recommended daily dosage for this composition can be two tablets per day for a total of 15 mg methylthioninium chloride, 20 mg of pyrroloquinoline quinone, and 1 mg tesofensine. In some examples, when forming compressed tablets, the tablets may be coated with a wax or polymer applied thereto. It is noted that the various components can be combined in liquid form and dried, or can be delivered as a liquid suspension.

### Example 17-Preparation of Liquid Nutritional Supplement Compositions (LNS1)

A liquid nutritional supplement composition (LNS1) is prepared by combining a 1:1 weight ratio of methylated vitamin B12 and vitamin C with an aqueous 1.5 vol % mineral blend including sodium, calcium, magnesium, potassium, and silicon. Each of these minerals is independently in the form of a mineral salt, a mineral oxide, or a mineral chloride. The liquid nutritional supplement composition formed is then divided into 30 ml bottles that contains about 28.4 grams of an aqueous liquid vehicle, e.g., water, about 1 gram of the mineral blend, about 0.3 grams of the methylated vitamin B, and about 0.3 grams of the vitamin C. In accordance with this, delivery of about 1 mL as a single dose of LNS1 to a subject is sufficient to delivery about 25 mg to about 40 mg of the mineral blend, about 10 mg of the methylated vitamin B12, and about 10 mg of the vitamin C. In accordance with examples of the present disclosure, some hydrogen peroxide may be added or formed in situ, e.g., from about 0.01 wt % to about 0.5 wt %, e.g., about 0.06 wt %.

### Example 18-Preparation of Liquid Nutritional Supplement Compositions (LNS2)

A liquid nutritional supplement composition (LNS2) is prepared by combining methylated vitamin B12 with an aqueous 1.5 vol % mineral blend including sodium, calcium, magnesium, potassium, and silicon. Each of these minerals is independently in the form of a mineral salt, a mineral oxide, or a mineral chloride. The liquid nutritional supplement composition formed is then divided into 30 mL bottles that contains about 28.55 grams of an aqueous liquid vehicle, e.g., water, about 1 gram of the mineral blend and about 0.45 grams of the methylated vitamin B. In accordance with this, delivery of about 1 mL as a single dose of LNS2 to a subject is sufficient to delivery about 25 mg to about 40 mg of the mineral blend, about 15 mg of the methylated vitamin B12. In accordance with examples of the present disclosure, some hydrogen peroxide may be added or formed in situ, e.g., from about 0.01 wt % to about 0.5 wt %, e.g., about 0.06 wt %.

### Example 19-Preparation of Liquid Nutritional Supplement Compositions (LNS3)

A liquid nutritional supplement composition (LNS3) is prepared by combining vitamin C with an aqueous 1.5 vol % mineral blend including sodium, calcium, magnesium, potassium, and silicon. Each of these minerals is independently in the form of a mineral salt, a mineral oxide, or a mineral chloride. The liquid nutritional supplement composition formed is then divided into 30 ml bottles that contains about 28.55 grams of an aqueous liquid vehicle, e.g., water, about 1 gram of the mineral blend and about 0.45 grams of the vitamin C. In accordance with this, delivery of about 1 mL as a single dose of LNS3 to a subject is sufficient to delivery about 25 mg to about 40 mg of the mineral blend, about 15 mg of the vitamin C. In accordance with examples of the present disclosure, some hydrogen peroxide may be added or formed in situ, e.g., from about 0.01 wt % to about 0.5 wt %, e.g., about 0.06 wt %.

### Example 20-HBOT Combined with Treatment of Subjects for COVID-19 (SARS-CoV-2 Virus) Using MB4

A clinical study was conducted to evaluate the combination of a liquid suspension (Liquid Suspension 1) of colloidal silver (Ag404) with admixed with a suspension of metal oxide and chlorides (containing magnesium chloride, potassium chlorate, sodium chloride, and magnesium oxide) and its effectiveness for killing COVID-19. The composition prepared was similar to that as prepared in accordance with Example 4 (MB4). Multiple aluminum surfaces (12×12 inches each) were used in the evaluation, which were cleaned and dried and then sprayed with a biological sample that contained the COVID-19 virus until each aluminum surface was well covered. Liquid Suspension 1 was then sprayed on the aluminum surfaces and then at various time periods, e.g., 2 minutes, 10 minutes, 6 hours, and 24 hours, each treated sample was used to generate smears taken from the center of each aluminum surface, which were then evaluated by a clinical technician using standard microbe software. An average microbial kill percentage was determined for each sample after each time frame, all of which exhibited about a 97-98% of the COVID-19 virus being killed. Thus, it was surmised that at about 2 minutes, most of the virus was killed, with only nominal increase (if any) when exposed to Liquid Suspension 1 for longer periods of time.

In treating the COVID-19 virus in a subject, past protocols have included intubation, which in some instances exacerbated poor outcomes, including death. As COVID-19 is often associated with diminishing oxygen content to the body (due in part to compromised lungs), combining the use of the composition of MB4 with hyperbaric oxygen therapy (HBOT) provides a more effective way of driving oxygen into body tissues, including the lung tissue, without the drawbacks and dangers associated with intubation of an infected subject.

### Example 21-Red-light Therapy Combined with MB1 for Cognitive Enhancement and Energy

A methylthioninium salt-containing composition (MB1) prepared in accordance with Example 1 is administered to a subject on a daily basis for ADD/ADHD, brain fog, and/or lack of energy. MB1 has shown efficacy in treating such conditions with multiple subjects utilizing various dosages suitable for each individual. In accordance with the present disclosure, administration of MB1 can be conducted in combination with red-light therapy for 30 minutes per day, five (5) times per week, at two peak wavelengths of red-light energy, e.g., about 660 nm and about 820 nm. Combining the administration of MB1 with red-light therapy can increase the efficacy of treatment.

### Example 22-Pulsed Electromagnetic Field (PEMF) Therapy Combined with MB5 for Cognitive Enhancement and Energy

A methylthioninium salt-containing composition (MB5) prepared in accordance with Example 5 is administered to a subject on a daily basis for ADD/ADHD, brain fog, and/or lack of energy and focus. MB5 has shown efficacy in treating such conditions with multiple subjects utilizing various dosages suitable for each individual. In accordance with the present disclosure, administration of MB5 can be conducted in combination with pulsed electromagnetic field therapy for 30 minutes per day, five (5) times per week, using a Schumann resonance pulse repetition frequency, e.g., about 7.8 Hz. Combining the administration of MB5 with PEMF therapy can increase the efficacy of treatment.

### Example 23-Hyperbaric Oxygen Therapy (HBOT) Combined with MB6 for Cognitive Enhancement and Energy

A methylthioninium salt-containing composition (MB6) prepared in accordance with Example 6 is administered to a subject on a daily basis for ADD/ADHD, brain fog, and/or lack of energy and focus. MB6 has shown efficacy in treating such conditions with multiple subjects utilizing various dosages suitable for each individual. In accordance with the present disclosure, administration of MB6 can be conducted in combination with hyperbaric oxygen therapy for 90 minutes per day, three (3) times per week, using at 99 vol % oxygen and about 1.5 ATA of pressure. Combining the administration of MB5 with HBOT can increase the efficacy of treatment.

### Example 24-Sauna Therapy and/or Cold Plunge Therapy Combined with MB2 for Weight Loss Support

A methylthioninium salt-containing composition (MB2) prepared in accordance with Example 2 is administered to multiple subjects on a daily basis for weight loss support (used along with GLP-1 agonist medication), while retaining mental performance and energy. Quantitative (weight loss) and qualitative (user comments) reported positive results. Various dosages were used suitable for each individual. In accordance with the present disclosure, administration of MB2 can be conducted in combination with infrared sauna therapy for 25 minutes at 60° C. (about 140° F.) immediately followed by cold plunge therapy for 5 minutes at 10° C. (about 50° F.). The MB2 composition is orally administered on a daily basis, and the sauna/cold plunge therapies are administered 3 or 4 times per week, which promotes additional metabolic boost and weight loss support.

### Example 25-Cold Plunge Therapy Combined with MB8 for Improved Mood, Energy, and Weight Loss Support

A 44-year-old male with reduced mood and energy, also having a strong appetite was given the methylthioninium salt-containing compound of Example 8 (MB8). After a period of 3 weeks with daily dosages of 30 mg methylene blue and 500 mg urolithin A (active ingredients as outlined in the composition example, but at 2 tablets per day), the subject reported significant improvement in overall mood and energy, with a more noticeable decrease in appetite. In accordance with the present disclosure, daily administration of MB8 can be conducted in combination with 3-5 cold plunge therapy sessions per week, each at about 3-5 minutes and about 12° C. (about 53.6° F.), further enhancing the metabolic boost provided by the daily administration of MB8.

### Example 26-Red-Light Therapy Combined with MB8 for Improved Mood, Energy, and Weight Loss Support

A 60-year-old male seeking improved stamina and decreased appetite cravings for processed foods was given the methylthioninium salt-containing compound of Example 8 (MB8). After 30 days with daily dosages of 30 mg methylene blue and 500 mg urolithin A (active ingredients as outlined in the composition example, but at 2 tablets per day), the subject reported a curbed appetite while eating and more efficient digestion and better body function, reporting a 5 pound weight loss in the 30 days without any change in daily activity or exercise. In accordance with the present disclosure, daily administration of MB8 can be conducted in combination with daily 30-minute red-light therapy sessions using various red-light therapy wavelengths of light, e.g., 630 nm, 660 nm, 800 nm, and 820 nm, further enhancing the metabolic boost provided by the daily administration of MB8.

### Example 27-Hyperbaric Oxygen Therapy (HBOT) Combined with MB8 for Enhanced Cognition and Reduced Brain Fog

A 72-year-old male was given the methylthioninium salt-containing compound of Example 8 (MB8) and reported results after about 2 weeks. The subject took daily dosages of 30 mg methylene blue and 500 mg urolithin A (active ingredients as outlined in the composition example, but at 2 tablets per day), and reported no adverse effects switching from a methylene blue compound without the urolithin A present, and furthermore, an improvement was noted due to the addition of the urolithin A. More specifically, the subject reported very calm and clear cognition that has been sustained. Furthermore, the subject reported that there was no discomfort nor any noticeable side effects of any kind, indicating he felt 10 years younger, e.g., felt like early 60s. In accordance with the present disclosure, administration of MB8 can be conducted in combination with hyperbaric oxygen therapy for 60 minutes per day, three (3) times per week, using at 99 vol % oxygen and about 2 ATA of pressure. Combining the administration of MB8 with HBOT can increase the efficacy of treatment.

### Example 28-Pulsed Electromagnetic Field (PEMF) Therapy and Red-Light Therapy Combined with MB8 for Enhanced Energy

A 54-year-old woman looking to boost her energy level was given the methylthioninium salt-containing compound of Example 8 (MB8) with daily dosages of 30 mg methylene blue and 500 mg urolithin A (active ingredients as outlined in the composition example, but at 2 tablets per day). The subject reported “incredible” results with much more energy, improving her exercise and biking routines. She reported being able to push her workouts further along with a significant reduction in appetite, making healthy food choices easier without feeling hungry or food deprived. Less time to go to sleep and waking up well rested and refreshed was also noted. In accordance with the present disclosure, administration of MB8 can be conducted in combination with pulsed electromagnetic field therapy and red-light therapy for 30 minutes per day, five (5) times per week. The device used for this combination therapy is configured to deliver PEMF therapy and/or red-light therapy at the same time. Frequencies selected for the PEMF therapy are from about 6 Hz to about 13 Hz and the wavelengths for the red-light therapy simultaneously deliver light at 660 nm and 820 nm. Combining the administration of MB8 with PEMF and red-light therapy can increase the efficacy of treatment.

### Example 29-Cold Plunge Therapy Combined with MB8 for Enhanced Energy During Workouts and Weight Loss/Control

A 56-year-old male skeptic of supplements who prioritized fitness (reporting 10% body fat) was given the methylthioninium salt-containing compound of Example 8 (MB8) and reported a six (6) pound weight loss and a positive impact on workout sessions. The subject also reported a surge in energy during exercise, making completing an exercise session easier. The subject also reported enhanced reserve of strength and stamina. The subject took daily dosages of 30 mg methylene blue and 1 mg tesofensine (active ingredients as outlined in the composition example, but at 2 tablets per day). In accordance with the present disclosure, daily administration of MB8 can be conducted in combination with 3-5 cold plunge therapy sessions per week, each at about 3-5 minutes and about 6° C. (about 42.8° F.), further enhancing workout energy and weight control provided by the daily administration of MB8.

### Example 30-Pulsed Electromagnetic Field (PEMF) Therapy Combined with MB6 for Enhanced Mitochondrial Function

Two methylthioninium salt-containing compositions (MB3 and MB6) prepared in accordance with Examples 3 and 6, respectively, are administered at different times of the day to a subject on a daily basis for enhanced mitochondrial function, improved energy production, reduced oxidative stress, and enhanced cellular repair. In this example, MB3 was modified with a small amount of black pepper. Black pepper extract contains piperine, a potent bioavailability enhancer that can increase the absorption of nutrients, inhibit enzymes that break certain nutrients down, increase the absorption of various nutrients and compounds by inhibiting enzymes that break them down and enhance the permeability of the intestinal lining. In accordance with the present disclosure, administration of MB6 can be conducted in the morning, delivering 30 mg of methylene blue, 100 mg of glycine, 100 mg of N-acetylcysteine, and 50 mg of niacinamide. Administration of 1 mL of MB3 can occur in the evening to further enhance the therapy. Furthermore, these compositions can be used in conjunction with pulsed electromagnetic field therapy for 30 minutes per day, five (5) times per week, using frequencies from about 6 Hz to about 13 Hz, for example. Combining the administration of MB3, MB6, and PEMF therapy can increase the efficacy of treatment.

### Example 31-Hyperbaric Oxygen Therapy (HBOT) Combined with MB2, MB3, and MB5 for General Health and Wellness

Three methylthioninium salt-containing compositions (MB2, MB3, and MB5) prepared in accordance with Examples 2, 3, and 5, respectively, are administered as follows: 1) Each morning, multiple MB2 tablets are administered that include, in total, 5 mg methylene blue, 114 mg guarana, 150 mg green tea, and 200 mg L-theanine; 2) 1 mL of MB3 is administered immediately following the administration of MB2; and 3) Each night before bed, multiple tablets are administered of MB5 that include, in total, 15 mg methylene blue and 75 mg vitamin C. In accordance with the present disclosure, administration of the compositions can be conducted in combination with hyperbaric oxygen therapy for 90 minutes per day, three (3) times per week, using at 99 vol % oxygen and about 1.5 ATA of pressure. Combining the administration of MB2, MB3, and MB5 with HBOT can increase the efficacy of treatment.

### Example 32-HBOT, Red-Light Therapy, and/or PEMF Therapy Combined with Treatment of Subjects for Enhanced Absorption of a Physiological Compounds Using LNS1, LNS2, or LNS3

One or LNS1, LNS2, or LNS3 is prepared in accordance with one of Examples 17-19 (also shown more generally in Table 4 above) to provide enhanced absorption of a methylthioninium salt and/or a secondary physiologically active compound, such as urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or the like. In accordance with the present disclosure, administration can include a single dose of one or more of the LNS1, LNS2, or LNS3 to be about 15 mg to about 50 mg of the mineral blend, and about 5 mg to about 20 mg of the vitamin C and/or about 5 mg to about 20 mg of the methylated vitamin B12. As an example, these compositions (co-administered as a single formulation or as multiple formulations) can be used in combination with: i) hyperbaric oxygen therapy for 90 minutes per day, three (3) times per week, using at 99 vol % oxygen and about 1.5 ATA of pressure; ii) red-light therapy for 30 minutes per day (daily) at about 630 nm, 660 nm, 800 nm, and/or 820 nm; and/or iii) pulsed electromagnetic field therapy for 30 minutes per day, five (5) times per week, using frequencies from about 6 Hz to about 13 Hz, for example.

It is to be understood that the above-referenced arrangements are illustrative of the application of principles of the present disclosure. Thus, while the present technology has been described herein in connection with the exemplary embodiments, it will be apparent to those of ordinary skill in the art that numerous modifications and alternative arrangements can be made without departing from the principles and concepts of the disclosure as set forth in the claims.

1. A physiological protection or enhancement system, comprising:
a methylthioninium salt-containing composition, comprising a methylthioninium salt and a selected from urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, or a combination thereof; and
a physiological therapy device for use in combination with the methylthioninium salt-containing composition, wherein the physiological therapy device is selected from a thermal therapy device, an electromagnetic therapy device, an oxygenation therapy device, or a combination thereof.

2. The physiological protection or enhancement system of claim 1, wherein the physiological therapy device includes the thermal therapy device and is selected from a cold plunge therapy device, a cryotherapy device, a sauna therapy device, a localized heat or cold therapy device, or a combination thereof.

3. The physiological protection or enhancement system of claim 1, wherein the physiological therapy device includes the electromagnetic therapy device and is selected from a red-light therapy device, a pulse electromagnetic field therapy device, an electric stimulation therapy device, or a combination thereof.

4. The physiological protection or enhancement system of claim 1, wherein the physiological therapy device includes the oxygenation therapy device and is selected from a hyperbaric oxygen therapy device, an enhanced oxygen therapy device, or a combination thereof.

5-6. (canceled)

7. The physiological protection or enhancement system of claim 1, wherein the secondary physiologically active compound includes:
the urolithin A at a methylthioninium salt to urolithin A weight ratio from about 1:600 to about 1:4,
the pyrroloquinoline quinone at a methylthioninium salt to pyrroloquinoline quinone weight ratio from about 1:20 about 50:1,
the tesofensine at a methylthioninium salt to tesofensine weight ratio from about 1:2 to about 100:1, or
a combination thereof.

8. The physiological protection or enhancement system of claim 7, wherein the methylthioninium salt-containing composition includes both the urolithin A and the pyrroloquinoline quinone, or wherein the methylthioninium salt-containing composition includes the tesofensine and one or both of the urolithin A or the pyrroloquinoline quinone.

9. (canceled)

10. The physiological protection or enhancement system of claim 7, wherein a single daily dose or divided daily dose of the methylthioninium salt-containing composition includes from about 1 mg to about 50 mg of the methylthioninium salt, and wherein the secondary physiologically active compound is selected from about 100 mg to about 1200 mg of the urolithin A, from about 0.5 mg to about 40 mg of the pyrroloquinoline quinone, from about 0.25 mg to about 4 mg of the tesofensine, or a combination thereof.

11. The physiological protection or enhancement system of claim 7, wherein the secondary physiologically active compound includes the urolithin A, the pyrroloquinoline quinone, and at least one of the guarana, the L-theanine, the green tea, the caffeine, the mineral oxides, the mineral chlorides, the N-acetylcysteine, the vitamin B3, the glycine, the vitamin C, or a combination thereof.

12-13. (canceled)

14. The physiological protection or enhancement system of claim 7, comprising the nicotinamide adenine dinucleotide in the form of NADH, NAD+, or a combination thereof at a methylthioninium salt and nicotinamide adenine dinucleotide weight ratio from about 1:100 to about 5:2.

15. (canceled)

16. The physiological protection or enhancement system of claim 1, wherein the secondary physiologically active compound includes:
the caffeine at a methylthioninium salt to caffeine weight ratio from about 1:75 to about 1:4,
the L-theanine at a methylthioninium salt and the L-theanine weight ratio from about 1:200 to about 1:10, or
a combination thereof.

17. The physiological protection or enhancement system of claim 16, comprising both the caffeine and the L-theanine.

18. The physiological protection or enhancement system of claim 16, wherein the secondary physiologically active compound includes:
the green tea at a methylthioninium salt to green tea weight ratio from about 1:3 to about 1:25,
the guarana at a methylthioninium salt to guarana weight ratio of about 1:10 to about 1:40, or
a combination thereof,
wherein the green tea, the guarana, or both are a source for at least a portion of the caffeine present in the stress or anxiety reducing compound.

19-21. (canceled)

22. The physiological protection or enhancement system of claim 1, wherein the methylthioninium salt-containing composition is in the form of a liquid suspension, and wherein the secondary physiologically active compound includes the mineral oxides and the mineral chlorides at a methylthioninium salt to mineral oxides and mineral chlorides weight ratio of about 100:1 to about 1:4, and wherein the methylthioninium salt-containing composition further includes hydrogen peroxide formed in situ.

23. The physiological protection or enhancement system of claim 22, wherein the liquid suspension further comprises colloidal silver, magnesium chloride, potassium chlorate, sodium chloride, magnesium oxide, the cannabidiol, the vitamin B6, the vitamin E, the manganese, the nicotine, the caffeine, the L-theanine, or a combination thereof.

24-29. (canceled)

30. The physiological protection or enhancement system of claim 1, wherein the secondary physiologically active compound includes the vitamin C at a methylthioninium salt to vitamin C weight ratio from about 1:2 to about 1:10.

31-33. (canceled)

34. The physiological protection or enhancement system of claim 1, wherein the secondary physiologically active compound includes:
the glycine at a methylthioninium salt to glycine weight ratio from about 1:3 to about 1:25,
the N-acetylcysteine at a methylthioninium salt to N-acetylcysteine weight ratio from about 1:3 to about 1:25,
the vitamin B3 at a methylthioninium salt to vitamin B3 weight ratio from about 1:2 to about 1:12, or
a combination thereof.

35. The physiological protection or enhancement system of claim 34, wherein the glycine and the N-acetylcysteine are both present at a weight ratio from about 1:2 to about 2:1.

36-39. (canceled)

40. The physiological protection or enhancement system of claim 1, wherein the secondary physiologically active compound includes:
the cannabinoid at a methylthioninium salt to cannabinoid weight ratio from about 1:3 to about 1:10,
the vitamin B6 at a methylthioninium salt to vitamin B6 weight ratio from about 10:1 to about 2:1,
the vitamin E at a methylthioninium salt to vitamin E weight ratio from about 1:3 to about 3:1,
the manganese at a methylthioninium salt to manganese metal content weight ratio from about 10:1 to about 2:1, or
a combination thereof.

41. The physiological protection or enhancement system of claim 40, wherein the secondary physiologically active compound further includes:
at least two of the cannabinoid, the vitamin B6, the vitamin E, or the manganese;
the nicotine, the caffeine, the L-theanine, or a combination thereof.

42-45. (canceled)

46. The physiological protection or enhancement system of claim 1, further comprising black pepper at a sufficient concentration to enhance absorption of the methylthioninium salt, the secondary physiologically active compound, or both.

47. (canceled)

48. The physiological protection or enhancement system of claim 1, wherein the physiological therapy device includes a red-light therapy device adapted to delivery red-light therapy to a surface of a subject at a wavelength from about 600 nm to about 750 nm, from greater than about 750 nm to about 1400 nm, or a combination thereof.

49. The physiological protection or enhancement system of claim 1, wherein the physiological therapy device includes a pulsed electromagnetic field device adapted to delivery pulsed electromagnetic field therapy at a pulse repetition frequency from greater than 0 Hz to about 50 Hz at a radiation intensity from about 10 milligauss to about 1000 gauss.

50. The physiological protection or enhancement system of claim 1, wherein the physiological therapy device includes a hyperbaric oxygen therapy device adapted to delivery oxygen from a pressurized oxygen-rich atmosphere to a subject at a pressure from about 1.25 ATA to about 4 ATA, wherein the oxygen-rich atmosphere includes from about 75 vol % oxygen to 100 vol % oxygen.

51-99. (canceled)

100. A physiological enhancement system, comprising:
a liquid nutritional supplement composition including from about 80 wt % to about 99 wt % liquid carrier (or water); from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and from about 0.1 wt % to about 5 wt % methylated vitamin B12, from about 0.1 wt % to about 5 wt % vitamin C, or both; and
a physiological therapy device for use in combination with the liquid nutritional supplement composition, wherein the physiological therapy device is selected from a thermal therapy device, an electromagnetic therapy device, an oxygenation therapy device, or a combination thereof.

101. The physiological enhancement system of claim 100, further comprising a secondary physiologically active compound having enhanced bioavailability in the presence of the mineral blend.

102. The physiological enhancement system of claim 101, wherein the secondary physiologically active compound includes a member selected from urolithin A, pyrroloquinoline quinone, tesofensine, nicotinamide adenine dinucleotide, caffeine, L-theanine, green tea, guarana, colloidal silver, glycine, N-acetylcysteine, vitamin B3, cannabidiol, vitamin B6, vitamin E, manganese, nicotine, GLP-1 agonist, or a combination thereof.

103. The physiological enhancement system of claim 100, further comprising a methylthioninium salt as part of the liquid nutritional supplement or as a separate dosage form.

104. The physiological enhancement system of claim 103, wherein the methylthioninium salt and the mineral blend are present at a weight ratio of about 100:1 to about 1:4.

105. The physiological enhancement system of claim 100, wherein the physiological therapy includes:
the electromagnetic therapy in the form of red-light therapy, pulse electromagnetic field therapy, or both,
the oxygenation therapy in the form of hyperbaric oxygen therapy, enhanced oxygen therapy, or a combination thereof,
the thermal therapy in the form of cold plunge therapy, cryotherapy, sauna therapy, localized heat or cold therapy, or both, or
a combination thereof.

106. A method of protecting or enhancing a physiological function, comprising:
orally administering multiple physiologically active compounds to a subject, wherein the multiple physiologically active compounds are administered as:
a liquid nutritional supplement composition including:
from about 80 wt % to about 99 wt % liquid carrier (or water);
from about 0.25 wt % to about 7.5 wt % mineral blend, wherein the mineral blend includes sodium, calcium, magnesium, potassium, and silicon each being independently present in the form of a mineral salt, a mineral oxide, or a mineral chloride; and
from about 0.1 wt % to about 5 wt % methylated vitamin B12, from about 0.1 wt % to about 5 wt % vitamin C, or both; or
a methylthioninium salt and a secondary physiologically active compound selected from urolithin A, pyrroloquinoline quinone, tesofensine, nicotinamide adenine dinucleotide, caffeine, L-theanine, green tea, guarana, mineral oxides, mineral chlorides, colloidal silver, vitamin C, glycine, N-acetylcysteine, vitamin B3, cannabidiol, vitamin B6, vitamin E, manganese, nicotine, the liquid nutritional supplement composition, or a combination thereof to a subject; and
subjecting the subject to a physiological therapy selected from thermal therapy, electromagnetic therapy, oxygenation therapy, or a combination thereof while the subject is dosed with clinically effective levels of the multiple physiologically active compounds to protect or enhance the physiological function.

107. The method of claim 106, further comprising administering at least the secondary physiologically active compound with the liquid nutritional supplement composition to promote having enhanced bioavailability of the secondary physiologically active compound in the presence of the mineral blend.

108. (canceled)

109. The method of claim 106, further comprising administering at least the methylthioninium salt as part of the liquid nutritional supplement composition or as a separate dosage form at a methylthioninium salt to mineral blend weight ratio from about 100:1 to about 1:4.

110. (canceled)

111. The method of claim 106, wherein the physiological therapy includes:
the electromagnetic therapy in the form of red-light therapy, pulse electromagnetic field therapy, or both,
the oxygenation therapy in the form of hyperbaric oxygen therapy, enhanced oxygen therapy, or a combination thereof,
the thermal therapy in the form of cold plunge therapy, cryotherapy, sauna therapy, localized heat or cold therapy, or both, or
a combination thereof.

112. The method of claim 111, wherein the physiological therapy includes the red-light therapy conducted for from about 5 minutes to about 60 minutes at a peak wavelength:
from about 600 nm to about 750 nm,
from greater than about 750 nm to about 1400 nm,
or a combination thereof.

113. The method of claim 111, wherein the physiological therapy includes the pulse electromagnetic field therapy conducted for from about 5 minutes to about 60 minutes at a pulse repetition frequency greater than 0 Hz to about 50 Hz at a radiation intensity from about 10 milligauss to about 1000 gauss.

114. The method of claim 111, wherein the physiological therapy includes the pulse electromagnetic field therapy conducted for from about 5 minutes to about 60 minutes at a pulse repetition frequency from about 1 Hz to about 40 Hz at a radiation intensity from about 200 milligauss to about 2 gauss.

115. The method of claim 111, wherein the physiological therapy includes the oxygenation therapy in the form of the hyperbaric oxygen therapy conducted for from about 15 minutes to about 2 hours in a hyperbaric chamber having a chamber pressure from greater than 1ATA to about 4 ATA with a chamber oxygen content from about 75 vol % to 100 vol %.

116. The method of claim 111, wherein the physiological therapy includes the cold plunge therapy for about 1 minutes to 10 minutes at a temperature from about 3° C. to about 16° C., or the cryotherapy for about 1 minute to about 5 minutes at a temperature from about −120° C. to about −200° C.

117. The method of claim 106, wherein the multiple physiologically active compounds include the methylthioninium salt and the secondary physiologically active compound.

118. The method of claim 117, wherein the methylthioninium salt and the secondary physiologically active compound are co-formulated together as a methylthioninium salt-containing composition.

119. The method of claim 117, wherein the methylthioninium salt and the secondary physiologically active compound are formulated as separate compositions and are co-administered as the separate compositions.

120. The method of claim 117, wherein the secondary physiologically active compound includes:
the urolithin A at a methylthioninium salt to urolithin A weight ratio from about 1:600 to about 1:4,
the pyrroloquinoline quinone at a methylthioninium salt to pyrroloquinoline quinone weight ratio from about 1:20 to about 50:1,
the tesofensine at a methylthioninium salt to tesofensine weight ratio from about 1:2 to about 100:1, or
a combination thereof.

121. The method of claim 120, further comprising orally co-administering nicotinamide adenine dinucleotide in the form of NADH, NAD+, or a combination thereof at a daily dosage from about 20 mg to about 200 mg.

122. The method of claim 117, wherein the methylthioninium salt is dosed on a daily basis at a sufficient concentration to:
stimulate ATP production and the urolithin A is dosed at a sufficient concentration to recognize or repair mitochondria damage,
stimulate ATP production and the pyrroloquinoline quinone is dosed at a sufficient concentration to enhance nicotinamide adenine dinucleotide production, mitochondrial mitophagy, or a combination thereof, or
stimulate ATP production and the tesofensine is dosed at a sufficient concentration to enhance cognition due to increased levels of dopamine, norepinephrine, serotonin, or a combination thereof in the brain, feelings of fullness and satisfaction by curbing appetite, or a combination thereof.

123. The method of claim 120, wherein the methylthioninium salt is dosed on a daily basis at about 1 mg to about 50 mg per day, and the secondary physiologically active compound is dosed on a daily basis at from about 100 mg to about 1200 mg of the urolithin A, from about 0.5 mg to about 40 mg of the pyrroloquinoline quinone, from about 0.25 mg to about 4 mg of the tesofensine, or a combination thereof.

124. The method of claim 117, wherein the secondary physiologically active compound is selected from:
caffeine at a methylthioninium chloride to caffeine weight ratio from about 1:75 to about 1:4,
L-theanine at a methylthioninium chloride to L-theanine weight ratio from about 1:200 to about 1:10, L-theanine, or
a combination thereof.

125. The method of claim 124, wherein the secondary physiologically active compound includes guarana, green tea, or a combination thereof as a source of the caffeine.

126. The method of claim 124, wherein the methylthioninium salt and the secondary physiologically active compound are dosed on a daily basis at sufficient concentrations to treat attention disorders selected from attention deficit hyperactivity disorder (ADHD) or attention deficit disorder (ADD), to enhance or sustain weight loss, or to enhance metabolism of glucagon-like peptide-1 (GLP-1).

127. The method of claim 124, wherein the methylthioninium salt is dosed on a daily basis at about 1 mg to about 50 mg per day, and the secondary physiologically active compound is dosed on a daily basis at from about 20 mg to about 450 mg of caffeine, from about 25 mg to about 900 mg of L-theanine, or a combination thereof.

128. The method of claim 106, wherein the secondary physiologically active compound administered with the methylthioninium compound includes the liquid nutritional supplement composition.

129. The method of claim 128, wherein the methylthioninium compound to mineral blend weight ratio is from about 100:1 to about 1:4, wherein hydrogen peroxide formed in situ, and wherein the liquid nutritional supplement composition is either co-formulated with the methylthioninium salt or is orally co-administered as a separate composition.

130. The method of claim 106, wherein the liquid nutritional supplement composition is orally administered and further comprises colloidal silver having an average particle size from about 3 nm to about 20 nm, magnesium chloride, or a combination thereof.

131. The method of claim 106, wherein the secondary physiologically active compound includes vitamin C at a methylthioninium salt to vitamin C weight ratio from about 1:2 o about 1:10.

132. The method of claim 106, wherein the secondary physiologically active compound includes glycine at a methylthioninium salt to glycine weight ratio from about 1:3 to about 1:25, N-acetylcysteine at a methylthioninium salt to N-acetylcysteine weight ratio from about 1:3 to about 1:25, vitamin B3 at a methylthioninium salt to vitamin B3 weight ratio from about 1:2 to about 1:12, or a combination thereof.

133. The method of claim 106, wherein the secondary physiologically active compound includes the cannabinoid at a methylthioninium salt to cannabinoid weight ratio from about 1:3 to about 1:10, the vitamin B6 at a methylthioninium salt to vitamin B6 weight ratio from about 10:1 to about 2:1, the vitamin E at a methylthioninium salt to vitamin E weight ratio from about 1:3 to about 3:1, the manganese at a methylthioninium salt to manganese metal content weight ratio from about 10:1 to about 2:1, or a combination thereof.
